SEQUENCE LISTING (212 sequences)

SEQ ID NO: 1 - DNA, 630 bp, Homo sapiens
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 2 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 3 - protein, 8 aa, Homo sapiens
RQRYLYTD

SEQ ID NO: 4 - protein, 8 aa, Homo sapiens
QRYLYTDD

SEQ ID NO: 5 - protein, 8 aa, Homo sapiens
RYLYTDDA

SEQ ID NO: 6 - protein, 8 aa, Homo sapiens
YLYTDDAQ

SEQ ID NO: 7 - protein, 8 aa, Homo sapiens
LYTDDAQQ

SEQ ID NO: 8 - protein, 8 aa, Homo sapiens
YTDDAQQT

SEQ ID NO: 9 - protein, 8 aa, Homo sapiens
TDDAQQTE

SEQ ID NO: 10 - protein, 8 aa, Homo sapiens
DDAQQTEA

SEQ ID NO: 11 - protein, 8 aa, Homo sapiens
DAQQTEAH

SEQ ID NO: 12 - protein, 8 aa, Homo sapiens
AQQTEAHL

SEQ ID NO: 13 - protein, 8 aa, Homo sapiens
QQTEAHLE

SEQ ID NO: 14 - protein, 8 aa, Homo sapiens
QTEAHLEI

SEQ ID NO: 15 - protein, 9 aa, Homo sapiens
RQRYLYTDD

SEQ ID NO: 16 - protein, 9 aa, Homo sapiens
QRYLYTDDA

SEQ ID NO: 17 - protein, 9 aa, Homo sapiens
RYLYTDDAQ

SEQ ID NO: 18 - protein, 9 aa, Homo sapiens
YLYTDDAQQ

SEQ ID NO: 19 - protein, 9 aa, Homo sapiens
LYTDDAQQT

SEQ ID NO: 20 - protein, 9 aa, Homo sapiens
YTDDAQQTE

SEQ ID NO: 21 - protein, 9 aa, Homo sapiens
TDDAQQTEA

SEQ ID NO: 22 - protein, 9 aa, Homo sapiens
DDAQQTEAH

SEQ ID NO: 23 - protein, 9 aa, Homo sapiens
DAQQTEAHL

SEQ ID NO: 24 - protein, 9 aa, Homo sapiens
AQQTEAHLE

SEQ ID NO: 25 - protein, 9 aa, Homo sapiens
QQTEAHLEI

SEQ ID NO: 26 - protein, 9 aa, Homo sapiens
RQRYLYTDD

SEQ ID NO: 27 - protein, 10 aa, Homo sapiens
QRYLYTDDAQ

SEQ ID NO: 28 - protein, 10 aa, Homo sapiens
RYLYTDDAQQ

SEQ ID NO: 29 - protein, 10 aa, Homo sapiens
YLYTDDAQQT

SEQ ID NO: 30 - protein, 10 aa, Homo sapiens
LYTDDAQQTE

SEQ ID NO: 31 - protein, 10 aa, Homo sapiens
YTDDAQQTEA

SEQ ID NO: 32 - protein, 10 aa, Homo sapiens
TDDAQQTEAH

SEQ ID NO: 33 - protein, 10 aa, Homo sapiens
DDAQQTEAHL

SEQ ID NO: 34 - protein, 10 aa, Homo sapiens
DAQQTEAHLE

SEQ ID NO: 35 - protein, 10 aa, Homo sapiens
AQQTEAHLEI

SEQ ID NO: 36 - protein, 8 aa, Homo sapiens
GAADQSPE

SEQ ID NO: 37 - protein, 8 aa, Homo sapiens
AADQSPES

SEQ ID NO: 38 - protein, 8 aa, Homo sapiens
ADQSPESL

SEQ ID NO: 39 - protein, 8 aa, Homo sapiens
DQSPESLL

SEQ ID NO: 40 - protein, 8 aa, Homo sapiens
QSPESLLQ

SEQ ID NO: 41 - protein, 8 aa, Homo sapiens
SPESLLQL

SEQ ID NO: 42 - protein, 8 aa, Homo sapiens
PESLLQLK

SEQ ID NO: 43 - protein, 8 aa, Homo sapiens
ESLLQLKA

SEQ ID NO: 44 - protein, 8 aa, Homo sapiens
SLLQLKAL

SEQ ID NO: 45 - protein, 8 aa, Homo sapiens
LLQLKALK

SEQ ID NO: 46 - protein, 8 aa, Homo sapiens
LQLKALKP

SEQ ID NO: 47 - protein, 8 aa, Homo sapiens
QLKALKPG

SEQ ID NO: 48 - protein, 8 aa, Homo sapiens
LKALKPGV

SEQ ID NO: 49 - protein, 9 aa, Homo sapiens
GAADQSPES

SEQ ID NO: 50 - protein, 9 aa, Homo sapiens
AADQSPESL

SEQ ID NO: 51 - protein, 9 aa, Homo sapiens
ADQSPESLL

SEQ ID NO: 52 - protein, 9 aa, Homo sapiens
DQSPESLLQ

SEQ ID NO: 53 - protein, 9 aa, Homo sapiens
QSPESLLQL

SEQ ID NO: 54 - protein, 9 aa, Homo sapiens
SPESLLQLK

SEQ ID NO: 55 - protein, 9 aa, Homo sapiens
PESLLQLKA

SEQ ID NO: 56 - protein, 9 aa, Homo sapiens
ESLLQLKAL

SEQ ID NO: 57 - protein, 9 aa, Homo sapiens
SLLQLKALK

SEQ ID NO: 58 - protein, 9 aa, Homo sapiens
LLQLKALKP

SEQ ID NO: 59 - protein, 9 aa, Homo sapiens
LQLKALKPG

SEQ ID NO: 60 - protein, 9 aa, Homo sapiens
QLKALKPGV

SEQ ID NO: 61 - protein, 10 aa, Homo sapiens
GAADQSPESL

SEQ ID NO: 62 - protein, 10 aa, Homo sapiens
AADQSPESLL

SEQ ID NO: 63 - protein, 10 aa, Homo sapiens
ADQSPESLLQ

SEQ ID NO: 64 - protein, 10 aa, Homo sapiens
DQSPESLLQL

SEQ ID NO: 65 - protein, 10 aa, Homo sapiens
QSPESLLQLK

SEQ ID NO: 66 - protein, 10 aa, Homo sapiens
SPESLLQLKA

SEQ ID NO: 67 - protein, 10 aa, Homo sapiens
PESLLQLKAL

SEQ ID NO: 68 - protein, 10 aa, Homo sapiens
ESLLQLKALK

SEQ ID NO: 69 - protein, 10 aa, Homo sapiens
SLLQLKALKP

SEQ ID NO: 70 - protein, 10 aa, Homo sapiens
LLQLKALKPG

SEQ ID NO: 71 - protein, 10 aa, Homo sapiens
LQLKALKPGV

SEQ ID NO: 72 - protein, 8 aa, Homo sapiens
FLCQRPDG

SEQ ID NO: 73 - protein, 8 aa, Homo sapiens
LCQRPDGA

SEQ ID NO: 74 - protein, 8 aa, Homo sapiens
CQRPDGAL

SEQ ID NO: 75 - protein, 8 aa, Homo sapiens
QRPDGALY

SEQ ID NO: 76 - protein, 8 aa, Homo sapiens
RPDGALYG

SEQ ID NO: 77 - protein, 8 aa, Homo sapiens
PDGALYGS

SEQ ID NO: 78 - protein, 8 aa, Homo sapiens
DGALYGSL

SEQ ID NO: 79 - protein, 8 aa, Homo sapiens
GALYGSLH

SEQ ID NO: 80 - protein, 8 aa, Homo sapiens
ALYGSLHF

SEQ ID NO: 81 - protein, 8 aa, Homo sapiens
LYGSLHFD

SEQ ID NO: 82 - protein, 8 aa, Homo sapiens
YGSLHFDP

SEQ ID NO: 83 - protein, 8 aa, Homo sapiens
GSLHFDPE

SEQ ID NO: 84 - protein, 9 aa, Homo sapiens
FLCQRPDGA

SEQ ID NO: 85 - protein, 9 aa, Homo sapiens
LCQRPDGAL

SEQ ID NO: 86 - protein, 9 aa, Homo sapiens
CQRPDGALY

SEQ ID NO: 87 - protein, 9 aa, Homo sapiens
QRPDGALYG

SEQ ID NO: 88 - protein, 9 aa, Homo sapiens
RPDGALYGS

SEQ ID NO: 89 - protein, 9 aa, Homo sapiens
PDGALYGSL

SEQ ID NO: 90 - protein, 9 aa, Homo sapiens
DGALYGSLH

SEQ ID NO: 91 - protein, 9 aa, Homo sapiens
GALYGSLHF

SEQ ID NO: 92 - protein, 9 aa, Homo sapiens
ALYGSLHFD

SEQ ID NO: 93 - protein, 9 aa, Homo sapiens
LYGSLHFDP

SEQ ID NO: 94 - protein, 9 aa, Homo sapiens
YGSLHFDPE

SEQ ID NO: 95 - protein, 10 aa, Homo sapiens
FLCQRPDGAL

SEQ ID NO: 96 - protein, 10 aa, Homo sapiens
LCQRPDGALY

SEQ ID NO: 97 - protein, 10 aa, Homo sapiens
CQRPDGALYG

SEQ ID NO: 98 - protein, 10 aa, Homo sapiens
QRPDGALYGS

SEQ ID NO: 99 - protein, 10 aa, Homo sapiens
RPDGALYGSL

SEQ ID NO: 100 - protein, 10 aa, Homo sapiens
PDGALYGSLH

SEQ ID NO: 101 - protein, 10 aa, Homo sapiens
DGALYGSLHF

SEQ ID NO: 102 - protein, 10 aa, Homo sapiens
GALYGSLHFD

SEQ ID NO: 103 - protein, 10 aa, Homo sapiens
ALYGSLHFDP

SEQ ID NO: 104 - protein, 10 aa, Homo sapiens
LYGSLHFDPE

SEQ ID NO: 105 - protein, 8 aa, Homo sapiens
QSEAHGLP

SEQ ID NO: 106 - protein, 8 aa, Homo sapiens
SEAHGLPL

SEQ ID NO: 107 - protein, 8 aa, Homo sapiens
EAHGLPLH

SEQ ID NO: 108 - protein, 8 aa, Homo sapiens
AHGLPLHL

SEQ ID NO: 109 - protein, 8 aa, Homo sapiens
HGLPLHLP

SEQ ID NO: 110 - protein, 8 aa, Homo sapiens
GLPLHLPG

SEQ ID NO: 111 - protein, 8 aa, Homo sapiens
LPLHLPGN

SEQ ID NO: 112 - protein, 8 aa, Homo sapiens
PLHLPGNK

SEQ ID NO: 113 - protein, 8 aa, Homo sapiens
LHLPGNKS

SEQ ID NO: 114 - protein, 8 aa, Homo sapiens
HLPGNKSP

SEQ ID NO: 115 - protein, 8 aa, Homo sapiens
LPGNKSPH

SEQ ID NO: 116 - protein, 8 aa, Homo sapiens
PGNKSPHR

SEQ ID NO: 117 - protein, 8 aa, Homo sapiens
GNKSPHRD

SEQ ID NO: 118 - protein, 8 aa, Homo sapiens
NKSPHRDP

SEQ ID NO: 119 - protein, 8 aa, Homo sapiens
KSPHRDPA

SEQ ID NO: 120 - protein, 8 aa, Homo sapiens
SPHRDPAP

SEQ ID NO: 121 - protein, 8 aa, Homo sapiens
PHRDPAPR

SEQ ID NO: 122 - protein, 8 aa, Homo sapiens
HRDPAPRG

SEQ ID NO: 123 - protein, 8 aa, Homo sapiens
RDPAPRGP

SEQ ID NO: 124 - protein, 8 aa, Homo sapiens
DPAPRGPA

SEQ ID NO: 125 - protein, 8 aa, Homo sapiens
PAPRGPAR

SEQ ID NO: 126 - protein, 8 aa, Homo sapiens
APRGPARF

SEQ ID NO: 127 - protein, 8 aa, Homo sapiens
PRGPARFL

SEQ ID NO: 128 - protein, 8 aa, Homo sapiens
RGPARFLP

SEQ ID NO: 129 - protein, 8 aa, Homo sapiens
GPARFLPL

SEQ ID NO: 130 - protein, 8 aa, Homo sapiens
PARFLPLP

SEQ ID NO: 131 - protein, 8 aa, Homo sapiens
ARFLPLPG

SEQ ID NO: 132 - protein, 8 aa, Homo sapiens
RFLPLPGL

SEQ ID NO: 133 - protein, 9 aa, Homo sapiens
QSEAHGLPL

SEQ ID NO: 134 - protein, 9 aa, Homo sapiens
SEAHGLPLH

SEQ ID NO: 135 - protein, 9 aa, Homo sapiens
EAHGLPLHL

SEQ ID NO: 136 - protein, 9 aa, Homo sapiens
AHGLPLHLP

SEQ ID NO: 137 - protein, 9 aa, Homo sapiens
HGLPLHLPG

SEQ ID NO: 138 - protein, 9 aa, Homo sapiens
GLPLHLPGN

SEQ ID NO: 139 - protein, 9 aa, Homo sapiens
LPLHLPGNK

SEQ ID NO: 140 - protein, 9 aa, Homo sapiens
PLHLPGNKS

SEQ ID NO: 141 - protein, 9 aa, Homo sapiens
LHLPGNKSP

SEQ ID NO: 142 - protein, 9 aa, Homo sapiens
HLPGNKSPH

SEQ ID NO: 143 - protein, 9 aa, Homo sapiens
LPGNKSPHR

SEQ ID NO: 144 - protein, 9 aa, Homo sapiens
PGNKSPHRD

SEQ ID NO: 145 - protein, 9 aa, Homo sapiens
GNKSPHRDP

SEQ ID NO: 146 - protein, 9 aa, Homo sapiens
NKSPHRDPA

SEQ ID NO: 147 - protein, 9 aa, Homo sapiens
KSPHRDPAP

SEQ ID NO: 148 - protein, 9 aa, Homo sapiens
SPHRDPAPR

SEQ ID NO: 149 - protein, 9 aa, Homo sapiens
PHRDPAPRG

SEQ ID NO: 150 - protein, 9 aa, Homo sapiens
HRDPAPRGP

SEQ ID NO: 151 - protein, 9 aa, Homo sapiens
RDPAPRGPA

SEQ ID NO: 152 - protein, 9 aa, Homo sapiens
DPAPRGPAR

SEQ ID NO: 153 - protein, 9 aa, Homo sapiens
PAPRGPARF

SEQ ID NO: 154 - protein, 9 aa, Homo sapiens
APRGPARFL

SEQ ID NO: 155 - protein, 9 aa, Homo sapiens
PRGPARFLP

SEQ ID NO: 156 - protein, 9 aa, Homo sapiens
RGPARFLPL

SEQ ID NO: 157 - protein, 9 aa, Homo sapiens
GPARFLPLP

SEQ ID NO: 158 - protein, 9 aa, Homo sapiens
PARFLPLPG

SEQ ID NO: 159 - protein, 9 aa, Homo sapiens
ARFLPLPGL

SEQ ID NO: 160 - protein, 10 aa, Homo sapiens
QSEAHGLPLH

SEQ ID NO: 161 - protein, 10 aa, Homo sapiens
SEAHGLPLHL

SEQ ID NO: 162 - protein, 10 aa, Homo sapiens
EAHGLPLHLP

SEQ ID NO: 163 - protein, 10 aa, Homo sapiens
AHGLPLHLPG

SEQ ID NO: 164 - protein, 10 aa, Homo sapiens
HGLPLHLPGN

SEQ ID NO: 165 - protein, 10 aa, Homo sapiens
GLPLHLPGNK

SEQ ID NO: 166 - protein, 10 aa, Homo sapiens
LPLHLPGNKS

SEQ ID NO: 167 - protein, 10 aa, Homo sapiens
PLHLPGNKSP

SEQ ID NO: 168 - protein, 10 aa, Homo sapiens
LHLPGNKSPH

SEQ ID NO: 169 - protein, 10 aa, Homo sapiens
HLPGNKSPHR

SEQ ID NO: 170 - protein, 10 aa, Homo sapiens
LPGNKSPHRD

SEQ ID NO: 171 - protein, 10 aa, Homo sapiens
PGNKSPHRDP

SEQ ID NO: 172 - protein, 10 aa, Homo sapiens
GNKSPHRDPA

SEQ ID NO: 173 - protein, 10 aa, Homo sapiens
NKSPHRDPAP

SEQ ID NO: 174 - protein, 10 aa, Homo sapiens
KSPHRDPAPR

SEQ ID NO: 175 - protein, 10 aa, Homo sapiens
SPHRDPAPRG

SEQ ID NO: 176 - protein, 10 aa, Homo sapiens
PHRDPAPRGP

SEQ ID NO: 177 - protein, 10 aa, Homo sapiens
HRDPAPRGPA

SEQ ID NO: 178 - protein, 10 aa, Homo sapiens
RDPAPRGPAR

SEQ ID NO: 179 - protein, 10 aa, Homo sapiens
DPAPRGPARF

SEQ ID NO: 180 - protein, 10 aa, Homo sapiens
PAPRGPARFL

SEQ ID NO: 181 - protein, 10 aa, Homo sapiens
APRGPARFLP

SEQ ID NO: 182 - protein, 10 aa, Homo sapiens
PRGPARFLPL

SEQ ID NO: 183 - protein, 10 aa, Homo sapiens
RGPARFLPLP

SEQ ID NO: 184 - protein, 10 aa, Homo sapiens
GPARFLPLPG

SEQ ID NO: 185 - protein, 10 aa, Homo sapiens
PARFLPLPGL

SEQ ID NO: 186 - protein, 8 aa, Homo sapiens
MVGPSQGR

SEQ ID NO: 187 - protein, 8 aa, Homo sapiens
VGPSQGRS

SEQ ID NO: 188 - protein, 8 aa, Homo sapiens
GPSQGRSP

SEQ ID NO: 189 - protein, 8 aa, Homo sapiens
PSQGRSPS

SEQ ID NO: 190 - protein, 8 aa, Homo sapiens
SQGRSPSY

SEQ ID NO: 191 - protein, 8 aa, Homo sapiens
QGRSPSYA

SEQ ID NO: 192 - protein, 8 aa, Homo sapiens
GRSPSYAS

SEQ ID NO: 193 - protein, 9 aa, Homo sapiens
MVGPSQGRS

SEQ ID NO: 194 - protein, 9 aa, Homo sapiens
VGPSQGRSP

SEQ ID NO: 195 - protein, 9 aa, Homo sapiens
GPSQGRSPS

SEQ ID NO: 196 - protein, 9 aa, Homo sapiens
PSQGRSPSY

SEQ ID NO: 197 - protein, 9 aa, Homo sapiens
SQGRSPSYA

SEQ ID NO: 198 - protein, 9 aa, Homo sapiens
QGRSPSYAS

SEQ ID NO: 199 - protein, 10 aa, Homo sapiens
MVGPSQGRSP

SEQ ID NO: 200 - protein, 10 aa, Homo sapiens
VGPSQGRSPS

SEQ ID NO: 201 - protein, 10 aa, Homo sapiens
GPSQGRSPSY

SEQ ID NO: 202 - protein, 10 aa, Homo sapiens
PSQGRSPSYA

SEQ ID NO: 203 - protein, 10 aa, Homo sapiens
SQGRSPSYAS

SEQ ID NO: 204 - protein, 15 aa, Homo sapiens
  Primer
LYTDDAQQTEAHLEI

SEQ ID NO: 205 - protein, 20 aa, Homo sapiens
GAADQSPESLLQLKALKPGV

SEQ ID NO: 206 - protein, 19 aa, Homo sapiens
FLCQRPDGALYGSLHFDPE

SEQ ID NO: 207 - protein, 35 aa, Homo sapiens
QSEAHGLPLHLPGNKSPHRDPAPRGPARFLPLPGL

SEQ ID NO: 208 - protein, 14 aa, Homo sapiens
MVGPSQGRSPSYAS

SEQ ID NO: 209 - DNA, 20 bp, Artificial Sequence
  Primer
ccttttgcgagagagatact

SEQ ID NO: 210 - DNA, 20 bp, Artificial Sequence
  Primer
cctttagtgttccctgctag

SEQ ID NO: 211 - DNA, 20 bp, Artificial Sequence
  Primer
gtcctctcctgcaattcggg

SEQ ID NO: 212 - DNA, 20 bp, Artificial Sequence
  Primer
cgtcccatcctccctgatct

(19) United States
(12) Patent Application Publication
(10) Pub. No.: US 2011/0002845 A1
(43) Pub. Date: Jan. 6, 2011

(54) METHODS OF TREATING, DIAGNOSING OR DETECTING FGF21-ASSOCIATED DISORDERS

(75) Inventor: Liu Cheng, Emeryville, CA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 12/600,853

(22) PCT Filed: May 20, 2008
(86) PCT No.: PCT/US2008/006481

§ 371 Date: Nov. 19, 2009

Related U.S. Application Data
(60) Provisional application No. 60/939,512, filed on May 22, 2007.

(51) Int. Cl. A61K 51/10; A61K 39/395; A61K 31/7105; A61K 31/711; A61K 31/713; G01N 33/574; G01N 33/53; A61P 35/00

(52) U.S. Cl. 424/1.49; 424/158.1; 514/44 R; 424/145.1; 424/133.1; 424/135.1; 424/139.1; 435/7.23; 435/7.21

(57) ABSTRACT

The invention provides, inter alia, methods for treating cancer and vascular disease, compositions for treating cancer and vascular disease, and methods and compositions for diagnosing and/or detecting cancer and vascular disease.

### FIELD OF THE INVENTION

The present invention relates to FGF21-associated disorders including cancers and vascular diseases. More particularly, the invention relates to methods for treating cancer and vascular diseases, compositions for treating cancer and vascular diseases, and methods and compositions for diagnosing and/or detecting cancer and vascular diseases.

### BACKGROUND OF THE INVENTION

Fibroblast growth factor 21 (FGF21) is a member of the FGF protein family (U.S. Pat. No. 6,716,626). The FGF proteins identified to date belong to a family of signaling molecules that regulate growth and differentiation of a variety of cell types. The significance of FGF proteins to human physiology and pathology relates in part to their roles in embryogenesis, in blood vessel development and growth, and in bone growth. The majority of FGF family members have been associated with cellular activities including mitosis, development, transformation, angiogenesis, and cell survival. Several members of the FGF family and their biological roles are described in Crossley et al., Development 121:439-451 (1995); Ohuchi et al., Development 124:2235-2244 (1997); Gemel et al., Genomics 35:253-257 (1996); and Ghosh et al., Cell Growth and Differentiation 7:1425-1434 (1996).

FGF21 has been shown to be a potent activator of glucose uptake in adipocytes, to protect animals from diet-induced obesity when over-expressed in transgenic mice, and to lower blood glucose and triglyceride levels when therapeutically administered to diabetic rodents (Kharitonenkov et al., Jour. of Clinical Invest. 115:1627-1635, 2005). Also, FGF21 was observed to stimulate tyrosine phosphorylation of FGFR-1 and FGFR-2 in 3T3-L1 adipocytes (Id).

Angiogenesis, the formation of new blood vessels from existing blood vessels, is essential for tumor growth and metastasis. Tumor angiogenesis is driven by angiogenic growth factors secreted by tumor cells. The growth factors basic Fibroblast Growth Factor (bFGF), Vascular Endothelial Growth Factor (VEGF), Transforming Growth Factor alpha (TGFα), Platelet Derived Growth Factor (PDGF), angiopoietin (Ang)-1, and Ang-2 have been shown to induce angiogenesis in a variety of experimental models (Eggert et al., Clin. Cancer Res. 6:1900-1908, 2000). Because of the role angiogenesis plays in tumor growth, inhibition of angiogenic growth factors such as these is a strategy for treatment of cancer.

Vascular diseases affect the circulatory system and include diseases such as coronary artery disease (CAD) and peripheral artery disease (PAD). CAD is the major cause of death in the United States, and studies have linked defects in angiogenesis to the progression of CAD (e.g., Matsunaga, et al., Am J Physiol Heart Circ Physiol 288: H2042-H2046, 2005). Studies in animal models of ischemia show that administration of angiogenic growth factors can augment nutrient perfusion through neovascularization. Clinical trials of therapeutic angiogenesis using growth factors such as VEGF and FGF in patients with end-stage CAD have shown increases in exercise time and reductions in anginal symptoms. Angiogenesis has also been shown to improve blood flow following cardiac infarction (Takeshita et al., J. Clin. Invest. 93:662-670, 1994). Thus angiogenic factors are promising therapeutics for the treatment of vascular disease.

To date, however, the role of FGF21 in cancer and vascular disease has not been fully elucidated. Accordingly there is a need to identify compositions and methods that modulate FGF21. The present invention is directed to these, as well as other, important needs.

### SUMMARY OF THE INVENTION

In some aspects, the present invention provides methods of treating cancer or a cancer symptom in a patient in need thereof comprising administering to the patient a therapeutically effective amount of an FGF21 inhibitor.

In some aspects, the present invention provides methods of modulating an FGF21-related activity in a patient comprising administering to the patient an amount of an FGF21 modulator effective to modulate the FGF21-related biological activity.

In some aspects, the present invention provides methods of identifying a patient susceptible to FGF21 therapy comprising detecting evidence of overexpression of FGF21 in a cell sample from the patient as compared to a control cell sample. The presence of evidence of overexpression of FGF21 in the sample is indicative of a patient who is a candidate for FGF21 therapy. The absence of evidence of FGF21 expression in the sample is indicative of a patient who is not a candidate for FGF21 therapy. Therapeutically effective amounts of FGF21 inhibitors are administered to the patient if the patient is a candidate for FGF21 therapy. Conventional cancer treatment regimens are employed if the patient is not a candidate for FGF21 therapy.

In some aspects, the present invention provides methods of modulating one or more activities in a cell that expresses FGF21, comprising contacting the cells with an amount of an FGF21 inhibitor effective to modulate the one or more activities.

In some aspects, the present invention provides methods for detecting a tumor in a patient comprising administering to the patient a composition comprising an FGF21 inhibitor linked to an imaging agent and detecting the localization of the imaging agent in the patient.

In some aspects, the present invention provides methods for inhibiting the interaction of two or more cells that express FGF21 in a patient or patient sample comprising administering a therapeutically effective amount of an FGF21 inhibitor to the patient.

In some aspects, the present invention provides methods of expressing an FGF21 antibody in a cell wherein the FGF21 antibody specifically binds to an epitope comprising a sequence selected from the group consisting of SEQ ID NOs:3-203. The methods comprise expressing a nucleic acid encoding the FGF21 antibody in the cell.

In some aspects, the present invention provides methods of identifying a cancer inhibitor, wherein the cancer is characterized by differential expression of FGF21 compared to a control. The methods comprise contacting a cell expressing FGF21 with a candidate compound and determining whether an FGF21-related activity is modulated. Modulation of the FGF21-related activity is indicative of a cancer inhibitor.

In some aspects, the present invention provides methods of identifying a cancer inhibitor, said cancer characterized by differential expression of FGF21 compared to a control. The methods comprise contacting a cell expressing FGF21 with a candidate compound and determining whether activity of a downstream marker of FGF21 is modulated. Modulation of the downstream marker is indicative of a cancer inhibitor.

In some aspects, the present invention provides methods for determining the susceptibility of a patient to an FGF21 inhibitor comprising detecting evidence of overexpression of FGF21 in a cancer sample of said patient compared to a control sample. Evidence of overexpression of FGF21 is indicative of the patient's susceptibility to the FGF21 inhibitor.

In some aspects, the present invention provides methods of purifying FGF21 protein from a sample comprising (a) providing an affinity matrix comprising an anti-FGF21 antibody bound to a solid support; (b) contacting the sample with the affinity matrix to form an affinity matrix-FGF21 protein complex; (c) separating the affinity matrix-FGF21 protein complex from the remainder of the sample; and (d) releasing FGF21 protein from the affinity matrix.

In some aspects, the present invention provides methods of delivering a cytotoxic agent or a diagnostic agent to one or more cells that express FGF21. The methods comprise exposing a cell that expresses FGF21 to an antibody conjugated to a cytotoxic agent or diagnostic agent.

In some aspects, the present invention provides methods for determining the effectiveness of a candidate FGF21 inhibitor. The methods comprise contacting FGF21-expressing cells with the candidate FGF21 inhibitor and determining whether a downstream marker of FGF21 is modulated. Modulation of the downstream marker indicates that the candidate FGF21 inhibitor is an effective anti-cancer medication.

In some aspects, the present invention provides methods of determining whether a cancer is an FGF21-related cancer comprising comparing FGF21 expression in cancer and control cells. Upregulation of FGF21 expression in the cancer cells as compared to the control cells indicates that the cancer is an FGF21-related cancer.

In some aspects, the present invention provides methods of determining whether a cancer is an FGF21-related cancer comprising contacting a cancer sample with an FGF21 inhibitor, and measuring an FGF21 downstream marker in the cancer sample. Modulation of the downstream marker in the presence of the inhibitor as compared to the downstream marker in the absence of the inhibitor indicates that the cancer is an FGF21-related cancer.

In some aspects, the present invention provides methods of treating a cancer patient comprising determining whether a cancer is an FGF21-related cancer and administering to the patient an FGF21 inhibitor if the patient has an FGF21-related cancer, or treating the patient with a conventional cancer treatment regimen if the patient does not have an FGF21-related cancer.

In some aspects, the present invention provides methods of treating vascular disease or symptoms of a vascular disease in a patient in need thereof comprising administering to the patient a therapeutically effective amount of an FGF21 activator.

In some aspects, the present invention provides methods of inducing angiogenesis comprising contacting a cell sample with an amount of an FGF21 activator effective to increase endothelial cell proliferation by at least 30% as compared to a control cell sample.

In some aspects, the present invention provides methods of identifying an inhibitor of a vascular disease. The methods comprise contacting a cell sample expressing FGF21 with a candidate compound and determining whether an FGF21-related activity is modulated. Modulation of the FGF21-related activity is indicative of an inhibitor of vascular disease.

In some aspects, the present invention provides methods of identifying an inhibitor of a vascular disease comprising contacting a cell expressing FGF21 with a candidate compound and determining whether a downstream marker of FGF21 is modulated. Modulation of the downstream marker is indicative of an inhibitor of a vascular disease.

In some aspects, the present invention provides methods for determining the effectiveness of a candidate FGF21 activator as a medication for a vascular disease comprising contacting a cell sample with the candidate FGF21 activator and determining whether a downstream FGF21 marker is increased. An increase of the downstream marker in the presence of the candidate activator as compared to the downstream marker in the absence of the candidate activator indicates that the candidate FGF21 activator is an effective medication for a vascular disease.

In some aspects, the present invention provides compositions comprising an Fibroblast Growth Factor 21 (FGF21) modulator and one or more pharmaceutically acceptable carriers.

In some aspects, the present invention provides compositions comprising an FGF21 modulator and one or more pharmaceutically acceptable carriers, wherein the FGF21 modulator is an isolated double-stranded RNA (dsRNA); an isolated oligonucleotide comprising at least 10 consecutive nucleotides of a sequence of SEQ ID NO:1; an antibody that binds an epitope in a domain of FGF21 selected from the group consisting of the signal peptide domain and the FGF receptor binding domain; a small molecule; a mimetic; a soluble receptor; or a decoy.

In some aspects, the present invention provides purified antibodies that specifically bind one or more epitopes of an FGF21 polypeptide, wherein the epitope is in the signal peptide domain or the FGF receptor binding domain.

In some aspects, the present invention provides an isolated dsRNA molecule comprising a first strand of nucleotides comprising at least 19 consecutive nucleotides of a sequence of SEQ ID NO:1, and a second strand of nucleotides comprising a sequence substantially or completely complementary to the first strand, wherein the dsRNA molecule is less than 627 nucleotides long.

In some aspects, the present invention provides an isolated nucleic acid comprising at least 10 consecutive nucleotides of a sequence set forth in SEQ ID NO:1.

In some aspects, the present invention provides a fragment of a FGF21 polypeptide comprising between 10 and 209 contiguous amino acid residues of SEQ ID NO:2, wherein the fragment retains an FGF21 polypeptide activity.

In some aspects, the present invention provides compositions comprising an inhibitor of an FGF21 receptor and one or more pharmaceutically acceptable carriers, wherein the inhibitor is an isolated double-stranded RNA (dsRNA); an isolated oligonucleotide comprising at least 10 consecutive nucleotides of a sequence of SEQ ID NO:1; an antibody that binds an epitope in a domain of an FGF21 receptor; a small molecule; a mimetic; a soluble receptor; or a decoy.

These and other aspects of the present invention will be elucidated in the following detailed description of the invention.

### BRIEF DESCRIPTION OF THE DRAWINGS

FIG. 1 depicts stimulation of endothelial cell proliferation by FGF21.

FIG. 2 depicts expression of FGF21 mRNA in cancer cells.

FIG. 3 depicts expression of FGF21 mRNA in normal cells.

FIG. 4 depicts an alignment between FGF19x and FGF21 full-length polypeptides.

### DETAILED DESCRIPTION

The present invention provides methods for treating FGF21-related disorders, including cancer and vascular diseases, compositions for treating FGF21-related disorders, and methods and compositions for diagnosing and/or detecting FGF21-related disorders.

The inventors of the present application have discovered, inter alia, that FGF21 is over-expressed in colon cancer cells, and has restricted expression in normal tissues. The inventors have also discovered that FGF21 stimulates cell proliferation of endothelial cells. Since endothelial cell proliferation is an essential step in angiogenesis, FGF21 is also useful for promoting new blood vessel growth, which can be used for the treatment of vascular diseases, such as CAD and PAD. In view of the observed effect of FGF21 on cell proliferation and the observed upregulation of FGF21 in colon cancer cells, FGF21 may be a useful target for the treatment of cancer. These and other aspects of the present invention are provided in the present application.

### DEFINITIONS

Various definitions are used throughout this document. Most words have the meaning that would be attributed to those words by one skilled in the art. Words specifically defined either below or elsewhere in this document have the meaning provided in the context of the present invention as a whole and as are typically understood by those skilled in the art.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); and Sambrook et al., Molecular Cloning: A Laboratory Manual (2nd Edition, 1989).

As used herein, the singular forms “a,” “an” and “the” include plural references unless the content clearly dictates otherwise. Thus, for example, reference to “an antibody” includes a mixture of two or more such antibodies.

As used herein, the term “about” refers to +/−20%, +/−10%, or +/−5% of a value.

As used herein, the term “FGF21” refers to a member of the fibroblast growth factor (FGF) protein family. An exemplary mRNA sequence of FGF21 (GenBank Accession No. NM_{—}019113) is set forth as SEQ ID NO:1, and an exemplary amino acid sequence of FGF21 (GenBank Accession No. NP_{—}061986) is set forth as SEQ ID NO:2.

As used herein, the term “FGF21 receptor” refers to a membrane-bound receptor for FGF21. Binding of FGF21 to FGF21 receptor results in a cellular response and/or activity, such as a cell signaling event. In some embodiments the FGF21 receptor is FGFR-1 or FGFR-2.

The terms “polypeptide” and “protein”, are used interchangeably and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “individual”, “subject”, “host” and “patient” are used interchangeably and refer to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, “cancer” refers to primary or metastatic cancers, leukemias, or lymphomas. The term “cancer cells” refers to cells that are transformed. These cells can be isolated from a patient who has cancer, or be cells that are transformed in vitro to become cancerous. Cancer cells can be derived from many types of samples including any tissue or cell culture line. In some embodiments the cancer cells are hyperplasias, tumor cells, or neoplasms. In some embodiments, the cancer cells are isolated from colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma and melanoma. In some embodiments, the cancer cells are taken from established cell lines that are publicly available. In some embodiments, cancer cells are isolated from pre-existing patient samples or from libraries comprising cancer cells. In some embodiments, cancer cells are isolated and then implanted in a different host, e.g., in a xenograft. In some embodiments cancer cells are transplanted and used in a SCID mouse model. In some embodiments, the cancer is colon cancer. As used herein, the term “colon cancer” is used interchangeably with “rectal cancer” and “colorectal cancer,” and refers to a cancer that originates in the colon or rectum.

As used herein, the term “transformed” refers to any alteration in the properties of a cell that is stably inherited by its progeny. In some embodiments, “transformed” refers to the change of normal cell to a cancerous cell, e.g., one that is capable of causing tumors. In some embodiments, a transformed cell is immortalized. Transformation can be caused by a number of factors, including overexpression of a receptor in the absence of receptor phosphorylation, viral infection, mutations in oncogenes and/or tumor suppressor genes, and/or any other technique that changes the growth and/or immortalization properties of a cell.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, or the like.

As used herein, the term “metastasis” refers to a cancer which has spread to a site distant from the origin of the cancer, e.g. from the primary tumor. Sites of metastasis include without limitation, the bone, lymph nodes, lung, liver, and brain.

As used herein, the term “angiogenesis” refers to the growth of new blood vessels from pre-existing vessels.

As used herein, the term “clinical endpoint” refers to a measurable event indicative of cancer. Clinical endpoints include without limitation, time to first metastasis, time to subsequent metastasis, size and/or number of metastases, size and/or number of tumors, location of tumors, aggressiveness of tumors, quality of life, pain and the like. Those skilled in the art are credited with the ability to determine and measure clinical endpoints. Methods of measuring clinical endpoints are known to those of skill in the art.

As used herein, the term “sample” refers to biological material from a patient. The sample assayed by the present invention is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized.

As used herein, the term “biological molecule” includes, but is not limited to, polypeptides, nucleic acids, and saccharides.

As used herein, the term “modulating” refers to a change in the quality or quantity of a gene, protein, or any molecule that is inside, outside, or on the surface of a cell. The change can be an increase or decrease in expression or level of the molecule. The term “modulates” also includes changing the quality or quantity of a biological function/activity including, without limitation, cell proliferation, growth, adhesion, apoptosis, intracellular signaling, cell-to-cell signaling, and the like.

As used herein, the term “modulator” refers to a composition that modulates one or more physiological or biochemical events associated with cancer or a vascular disease. In some embodiments the modulator inhibits one or more biological activities associated with cancer. In some embodiments the modulator increases one or more biological activities thereby ameliorating a symptom associated with vascular disease. In some embodiments the modulator is a small molecule, an antibody, a mimetic, a decoy or an oligonucleotide. In some embodiments the modulator acts by blocking ligand binding or by competing for a ligand-binding site. In some embodiments the modulator acts independently of ligand binding. In some embodiments the modulator does not compete for a ligand binding site. In some embodiments the modulator blocks expression of a gene product involved in cancer or a vascular disease. In some embodiments the modulator blocks a physical interaction of two or more biomolecules involved in cancer or a vascular disease. In some embodiments modulators of the invention inhibit one or more FGF21 biological activities selected from the group consisting of cell proliferation, angiogenesis, blood vessel formation, cell signaling, kinase activity, glucose uptake into adipocytes, cancer cell survival and apoptosis. In some embodiments modulators of the invention increase one or more FGF21 biological activities selected from the group consisting of cell proliferation, angiogenesis, blood vessel formation, cell signaling, kinase activity, glucose uptake into adipocytes, and apoptosis. Modulators of the invention may also inhibit interactions between FGF21 and an FGF21 receptor, or phosphorylation of an FGF21 receptor. The FGF21 receptor can be, for example, one or both of FGFR-1 or FGFR-2. In some embodiments, modulators of the invention increase interactions between FGF21 and an FGF21 receptor, and/or phosphorylation of an FGF21 receptor. In some embodiments the FGF21 modulator inhibits FGF21 expression. In some embodiments, the FGF21 modulator increases FGF21 expression.

A “gene product” is a biopolymeric product that is expressed or produced by a gene. A gene product may be, for example, an unspliced RNA, an mRNA, a splice variant mRNA, a polypeptide, a post-translationally modified polypeptide, a splice variant polypeptide etc. Also encompassed by this term are biopolymeric products that are made using an RNA gene product as a template (i.e. cDNA of the RNA). A gene product may be made enzymatically, recombinantly, chemically, or within a cell to which the gene is native. In some embodiments, if the gene product is proteinaceous, it exhibits a biological activity. In some embodiments, if the gene product is a nucleic acid, it can be translated into a proteinaceous gene product that exhibits a biological activity.

“Modulation of FGF21 activity,” as used herein, refers to an increase or decrease in FGF21 activity that can be a result of, for example, interaction of an agent with an FGF21 polynucleotide or polypeptide, inhibition of FGF21 transcription and/or translation (e.g., through antisense or siRNA interaction with the FGF21 gene or FGF21 transcript, through modulation of transcription factors that facilitate FGF21 expression), and the like. For example, modulation of a biological activity refers to an increase or a decrease in a biological activity. FGF21 activity can be assessed by means including, without limitation, assaying endothelial cell proliferation, assessing FGF21 polypeptide levels, or by assessing FGF21 transcription levels. Comparisons of FGF21 activity can also be accomplished by measuring levels of an FGF21 downstream marker, measuring inhibition of FGF21 signaling, measuring inhibition of FGF21 mediated cell adhesion, measuring activation of FGF21 mediated cancer cell apoptosis, measuring inhibition of cancer cell growth, measuring inhibition of tumor formation, and measuring inhibition of cyclin production. FGF21 activity can also be assessed by measuring angiogenesis, blood vessel formation, cell signaling, kinase activity, glucose uptake into adipocytes, interactions between FGF21 and an FGF21 receptor, or phosphorylation of an FGF21 receptor. In some embodiments the FGF21 receptor is FGFR-1 or FGFR-2, and phosphorylation of an FGF21 receptor can be tyrosine phosphorylation. In some embodiments modulation of FGF21 activity can cause modulation of an FGF21-related phenotype.

As used herein, the term “inhibit” refers to a reduction, decrease, inactivation or down-regulation of an activity or quantity. For example, in the context of the present invention, FGF21 modulators may inhibit one or more of cancer cell growth, tumor formation, cancer cell proliferation, cancer cell metastasis, cell migration, angiogenesis, FGF21 signaling, FGF21-mediated cell-cell adhesion, cell-cell interaction, FGF21-mediated cell-cell membrane interaction, and FGF21 expression. Inhibition may be at least 25%, at least 50%, at least 75%, at least 80%, at least 90%, at least 95%, at least 97%, at least 98%, at least 99%, or 100%, as compared to a control.

As used herein, the term “differentially expressed in a cancer cell” and “a polynucleotide that is differentially expressed in a cancer cell” are used interchangeably herein, and refer to a polynucleotide that represents or corresponds to a gene that is differentially expressed in a cancerous cell when compared with a cell of the same cell type that is not cancerous, e.g., mRNA is found at levels at least about 25%, at least about 50% to about 75%, at least about 90%, at least about 1.5-fold, at least about 2-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source, or between one cell in situ and a second cell removed from its tissue source. In some embodiments, the gene is upregulated in the cancer gene as compared to the normal cell.

An FGF21 associated-cancer is “inhibited” if at least one symptom or clinical endpoint of the cancer is alleviated, terminated, slowed, or prevented. As used herein, an FGF21 associated-cancer is also “inhibited” if recurrence or metastasis of the cancer is reduced, slowed, delayed, or prevented.

As used herein, the phrase “inhibits FGF21 mediated cell adhesion” refers to inhibition or abolition of cell-to-cell adhesion in the presence of an FGF21 inhibitor wherein at least one cell differentially expresses FGF21. In this context, FGF21 mediated cell adhesion can be decreased by FGF21 inhibitor at least 25%, at least 50%, at least 75%, at least 85%, at least 90%, at least 95%, up to 100% relative to FGF21 mediated cell adhesion in the absence of an FGF21 inhibitor. Comparisons of FGF21 mediated cell adhesion can be accomplished by measuring, for example, by labeling the cells of interest, incubating them with a population of unlabeled cells adhering to a substrate, and washing to separate the adherent from the non-adherent populations. In this manner, cell adhesion is determined by measuring the amount of label retained on the substrate. Examples of assay systems include, but are not limited to labeling with fluorescent probes such as calcein AM, CFMDA (5-chloromethylfluorescein diacetate), 5(6)-CFDA-SE [5-(and-6)-carboxyfluorescein diacetate, succinimidyl ester] and measuring fluorescence in fluorescence plate reader or via flow cytometry.

As used herein, the phrase “increasing cancer cell apoptosis” refers to increasing apoptosis of cancer cells that differentially express FGF21 in the presence of an FGF21 inhibitor. In this context, cancer cell apoptosis can be increased by an FGF21 inhibitor at least 25%, at least 50%, at least 75%, at least 85%, at least 90%, at least 95%, up to 100% relative to cancer cell apoptosis in the absence of an FGF21 inhibitor. Comparisons of cancer cell apoptosis can be accomplished by measuring, for example, DNA fragmentation, caspase activity, loss of mitochondrial membrane potential, increased production of reactive oxygen species (ROS), intracellular acidification, chromatin condensation, phosphatidyl serine (PS) levels at the cell surface, and increased cell membrane permeability.

DNA fragmentation can be measured, for example, with the TUNEL assay (terminal deoxynucleotide transferase dUTP nick end labeling). Commercial versions of the assay are widely available, for example, APO-BrdU™ TUNEL Assay Kit (Invitrogen), APO-DIRECT™ Kit (BD Biosciences Pharmingen) and ApoAlert™ DNA Fragmentation Assay Kit (Clontech, a Takara Bio Company).

Caspase activity can be monitored via fluorogenic, chromogenic and luminescent substrates specific for particular caspases. Commercial assay kits are available for at least caspases 1, 2, 3, 6, 7, 8 and 9. (See, for example, Invitrogen, Chemicon, CalBiochem, BioSource International, Biovision).

Loss of mitochondrial membrane potential can be measured with fluorescent dyes that differentially accumulate in healthy active mitochondria. One non-limiting example is the MitoTracker Red system from Invitrogen.

Production of reactive oxygen species (ROS) can be measured with fluorescent dyes including, for example, H2DCFDA (Invitrogen).

Intracellular acidification can be measured with fluorescent or chromogenic dyes.

Chromatin condensation can be measured with fluorescent dyes including, for example, Hoechst 33342.

Phosphatidyl serine (PS) levels can be measured at the cell surface. For example, Annexin V has a high affinity for PS. Numerous commercially available assays are suitable to monitor the binding of labeled AnnexinV to the cell surface.

Cell membrane permeability can be measured using dyes, such as the fluorescent dye, YO-PRO-1 (Invitrogen) which can enter apoptotic, but not necrotic cells.

As used herein, the phrase “inhibits cancer cell growth” refers to inhibition or abolition of cancer cell growth in the presence of an FGF21 inhibitor wherein the cell differentially expresses FGF21. In this context, cancer cell growth can be decreased by FGF21 inhibitor at least 25%, at least 50%, at least 75%, at least 85%, at least 90%, at least 95%, up to 100% relative to cancer cell growth in the absence of an FGF21 inhibitor. Comparisons of cancer cell growth can be accomplished using, for example, MTT assay (for example, the Vybrant® MTT Cell Proliferation Assay Kit (Invitrogen)); BrdU incorporation (for example, the Absolute-S SBIP assay (Invitrogen)); measuring intracellular ATP levels (for example using ATPLite™-M, 1,000 Assay Kit (PerkinElmer) or ATP Cell Viability Assay Kit (BioVision)); DiOc18 assay, a membrane permeable dye (Invitrogen); Glucose-6-phosphate dehydrogenase activity assay (for example, the Vibrant cytotoxicity assay (Invitrogen)); or measuring cellular LDH activity.

As used herein, the phrase “inhibits tumor formation” refers to inhibition or abolition of tumor formation in the presence of an FGF21 inhibitor wherein the tumor comprises cells that differentially express FGF21. In this context, tumor formation can be decreased by an FGF21 inhibitor at least 25%, at least 50%, at least 75%, at least 85%, at least 90%, at least 95%, and up to 100% relative to tumor formation in the absence of an FGF21 inhibitor. Comparisons of tumor formation can be accomplished using, for example, cell based assays (for example colony formation in soft agar); in vivo models of tumor formation typically relying upon injecting the cells of interest into animals (for example, athymic mice or rats, irradiated mice or rats; inoculation into immunologically privileged sites such as brain, cheek pouch or eye; inoculation of syngeneic animals), and monitoring the size of the mass after a defined time period.

As used herein, the phrase “inhibits cyclin D1” refers to the inhibition or abolition of FGF21 mediated cyclin production. In this context, FGF21 mediated cyclin production can be decreased by an inhibitory agent at least 25%, at least 50%, at least 75%, at least 85%, at least 90%, at least 95%, up to 100% relative to FGF21 mediated cyclin production in the absence of an FGF21 inhibitor. Comparisons of cyclin production can be accomplished by measuring, for example, cyclin mRNA levels via RT-PCR or northern blotting; cyclin polypeptide levels via immunoblotting, immunoprecipitation or ELISA; or using functional assays, including co-immunoprecipitation assays to measure levels of cyclin that are complexed with cyclin regulators such as cyclin-dependent kinases (CDK's) using for example antibodies that target CDK, p21WAF1, p27 KIP-1; and measuring phosphorylation of cyclins by the CDK's can be assayed through radiolabeling and immunoprecipitation analysis or FRET-based methods, for example, CDK2/Cyclin A Assay Kit (Molecular Devices).

As used herein, the phrase “inhibits phosphorylation of an FGF21 receptor” refers to the inhibition or abolition of FGF21 mediated FGF21 receptor phosphorylation. In this context, FGF21 mediated FGF receptor phosphorylation can be decreased by an inhibitory agent at least 25%, at least 50%, at least 75%, at least 85%, at least 90%, at least 95%, up to 100% relative to FGF21 mediated FGF21 receptor phosphorylation in the absence of an FGF21 inhibitor. Phosphorylation levels can be assessed using phosphorylation assays known to those of skill in the art. FGFR-1 and FGFR-2 are exemplary FGF21 receptors.

As used herein, the phrase “inhibits cell signaling” refers to decreasing the effect of FGF21 on downstream members of cellular signaling cascades that include FGF21. Cellular signaling cascades that include FGF21 are mediated by interactions between FGF21 and an FGF21 receptor such as FGFR-1 or FGFR-2. Inhibition of FGF21 signaling can be determined by measuring polypeptide or polynucleotide levels of downstream members of the cellular signaling pathway. For example, a downstream marker can be an FGF21 receptor, such as FGFR-1 or FGFR-2 gene expression. Those of skill in the art are credited with the ability of measuring FGF21 polypeptide and/or polynucleotide levels. The art-skilled can also measure levels of FGF21 downstream markers.

As used herein, the phrase “inhibits cell-cell interaction” refers to reducing or eliminating an interaction between two or more cells that express FGF21. In some embodiments, the interaction between the cells leads to a cell signal. Cell-cell interaction can be detected via a number of methods known to those of skill in the art, including, without limitation, the observation of membrane exchange between co-cultured, pre-labeled cells, labeled, for example, with different fluorescent membrane stains including PKH26 and PKH67 (Sigma).

A “FGF21 downstream marker”, as used herein, is a gene or gene product, or measurable indicia of a gene or gene product. In some embodiments, a gene or activity that is a downstream marker of FGF21 exhibits an altered level of expression in a cancer tissue or cancer cell (compared to its expression level in normal or healthy tissue), or in a vascular tissue. In some embodiments, an activity of the downstream marker is altered in the presence of an FGF21 modulator. In some embodiments, the downstream markers exhibit altered levels of expression when FGF21 is perturbed with an FGF21 modulator of the present invention. FGF21 downstream markers include, without limitation, FGFR-1 and FGFR-2.

As used herein, the term “vascular disease” refers to a disease of the circulatory system. Vascular diseases included arterial diseases, such as coronary artery disease (CAD), peripheral artery disease (PAD), abdominal aortic aneurysm; and venous diseases, such as blood clots, deep vein thrombosis, venous stasis disease, phlebitis, varicose veins. Atherosclerosis is the underlying cause of most vascular diseases, and thus a subject having atherosclerosis is a candidate for treatment with the compositions and methods described herein.

As used herein, the term “up-regulates” refers to an increase, activation or stimulation of an activity or quantity. Similarly, an FGF21 “activator” is a composition which up-regualates an FGF21 activity or quantity. For example, in the context of the present invention, FGF21 modulators may increase the level of an FGF21 receptor. In one embodiment, one or both of FGFR-1 or FGFR-2 may be upregulated in response to an FGF21 modulator. Upregulation can also refer to an FGF21-related activity, such as cell proliferation, angiogenesis, blood vessel formation, cell signaling, kinase activity, cancer cell survival, glucose uptake into adipocytes, interactions between FGF21 and an FGF21 receptor, or phosphorylation of an FGF21 receptor. The FGFR21 receptor can be one or both of FGFR-1 or FGFR-2. Up-regulation may be at least 25%, at least 50%, at least 75%, at least 100%, at least 150%, at least 200%, at least 250%, at least 400%, or at least 500% as compared to a control. FGF21 modulators may increase phosphorylation of an FGF21 receptor or increase angiogenesis.

As used herein, the term “N-terminus” refers to at least the first 10 amino acids of a protein.

As used herein, the terms “N-terminal domain” and “N-terminal region” are used interchangeably and refer to a fragment of a protein that begins at the first amino acid of the protein and ends at any amino acid in the N-terminal half of the protein. For example, the N-terminal domain of FGF21 may span from amino acid 1 of SEQ ID NO:2 to any amino acid between about amino acids 9 and 209 of SEQ ID NO:2.

As used herein, the term “C-terminus” refers to at least the last 10 amino acids of a protein.

As used herein, the terms “C-terminal domain” and “C-terminal region” are used interchangeably and refer to a fragment of a protein that begins at any amino acid in the C-terminal half of the protein and ends at the last amino acid of the protein. For example, the C-terminal domain of FGF21 begins at any amino acid from amino acid 105 to about amino acid 200 of SEQ ID NO:2 and ends at amino acid 209 of SEQ ID NO:2.

The term “domain” as used herein refers to a structural part of a biomolecule that contributes to a known or suspected function of the biomolecule. Domains may be co-extensive with regions or portions thereof and may also incorporate a portion of a biomolecule that is distinct from a particular region, in addition to all or part of that region.

As used herein, the term “signal domain” (also called “signal sequence” or “signal peptide”) refers to a peptide domain that resides in a continuous stretch of amino acid sequence at the N-terminal region of a precursor protein (often a membrane-bound or secreted protein) and is involved in post-translational protein transport. In many cases the signal domain is removed from the full-length protein by specialized signal peptidases after the sorting process has been completed. Each signal domain specifies a particular destination in the cell for the precursor protein. An exemplary signal domain of FGF21 is represented by amino acids 1-28 of SEQ ID NO:2 (see GenPept Accession No. NP_{—}061986).

As used herein, the term “receptor binding domain” refers to any portion or region of a protein that contacts a membrane-bound receptor protein, resulting in a cellular response, such as a signaling event.

As used herein, the term “ligand binding domain” refers to any portion or region of a protein retaining at least one qualitative binding activity of a corresponding native sequence of FGF21.

The term “region” refers to a physically contiguous portion of the primary structure of a biomolecule. In the case of proteins, a region is defined by a contiguous portion of the amino acid sequence of that protein. In some embodiments a “region” is associated with a function of the biomolecule.

The term “fragment” as used herein refers to a physically contiguous portion of the primary structure of a biomolecule. In the case of proteins, a portion is defined by a contiguous portion of the amino acid sequence of that protein and refers to at least 3-5 amino acids, at least 8-10 amino acids, at least 11-15 amino acids, at least 17-24 amino acids, at least 25-30 amino acids, and at least 30-45 amino acids. In the case of oligonucleotides, a portion is defined by a contiguous portion of the nucleic acid sequence of that oligonucleotide and refers to at least 9-15 nucleotides, at least 18-30 nucleotides, at least 33-45 nucleotides, at least 48-72 nucleotides, at least 75-90 nucleotides, and at least 90-130 nucleotides. In some embodiments, portions of biomolecules have a biological activity. In the context of the present invention, FGF21 polypeptide fragments do not comprise the entire FGF21 polypeptide sequence set forth in SEQ ID NO:2.

As used herein, the phrase “FGF21-related cells/tumors/samples” and the like refers to cells, samples, tumors or other pathologies that are characterized by differential expression of FGF21 relative to non-cancerous and/or non-metastatic cells, samples, tumors, or other pathologies. In some embodiments, FGF21-related cells, samples, tumors or other pathologies are characterized by increased evidence of FGF21 expression relative to non-metastatic cells, samples, tumors, or other pathologies.

As used herein, the term “antibody” refers to monoclonal and polyclonal antibodies, single chain antibodies, chimeric antibodies, bifunctional/bispecific antibodies, humanized antibodies, human antibodies, and complementary determining region (CDR)-grafted antibodies, that are specific for the target protein or fragments thereof. The term “antibody” further includes in vivo therapeutic antibody gene transfer. Antibody fragments, including Fab, Fab′, F(ab′)2, scFv, and Fv are also provided by the invention.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Antibody fragments” comprise a portion of an intact antibody, preferably comprising the antigen-binding or variable region thereof. Examples of antibody fragments include Fab, Fab′, F(ab′)2, and Fv fragments; diabodies; linear antibodies (Zapata et al., Protein Eng. 8(10): 1057-1062 [1995]); single-chain antibody molecules; and multispecific antibodies formed from antibody fragment(s).

An “intact” antibody is one that comprises an antigen-binding variable region as well as a light chain constant domain (C_{L}) and heavy chain constant domains, C_{H1}, C_{H2} and C_{H3}. The constant domains may be native sequence constant domains (e.g. human native sequence constant domains) or amino acid sequence variants thereof. In some embodiments, the intact antibody has one or more effector functions.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Human effector cells” are leukocytes that express one or more FcRs and perform effector functions. In some embodiments, the cells express at least FcγRIII and perform ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

As used herein, the term “epitope” refers to an antigenic determinant of a polypeptide. In some embodiments an epitope may comprise 3 or more amino acids in a spatial conformation which is unique to the epitope. In some embodiments epitopes are linear or conformational epitopes. Generally an epitope consists of at least 4, at least 6, at least 8, at least 10, and at least 12 such amino acids, and more usually, consists of at least 8-10 such amino acids. Methods of determining the spatial conformation of amino acids are known in the art, and include, for example, x-ray crystallography and 2-dimensional nuclear magnetic resonance.

As used herein, the term “epitope bearing fragment” refers to a fragment of a polypeptide that includes one or more epitopes. In some embodiments, the epitope bearing fragment is not the full-length polypeptide.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu. One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which are incorporated herein by reference.

The term “antagonist” is used in the broadest sense, and includes any molecule that partially or fully blocks, inhibits, or neutralizes a biological activity of a tumor cell antigen disclosed herein. In a similar manner, the term “agonist” is used in the broadest sense and includes any molecule that mimics a biological activity of a tumor cell antigen disclosed herein. Suitable agonist or antagonist molecules specifically include agonist or antagonist antibodies or antibody fragments, fragments or amino acid sequence variants of tumor cell antigens, peptides, antisense oligonucleotides, small organic molecules, etc. Methods for identifying agonists or antagonists of a tumor cell antigen may comprise contacting a tumor cell expressing the antigen of interest with a candidate agonist or antagonist molecule and measuring a detectable change in one or more biological activities normally associated with the tumor cell antigen. The antagonist may also be a peptide generated by rational design or by phage display (see, e.g., WO98/35036 published 13 Aug. 1998). In some embodiments, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

As used herein, the term “oligonucleotide” refers to a series of linked nucleotide residues. Oligonucleotides include without limitation, antisense and siRNA oligonucleotides. Oligonucleotides comprise portions of a DNA sequence and have at least about 10 nucleotides and as many as about 500 nucleotides. In some embodiments oligonucleotides comprise from about 10 nucleotides to about 50 nucleotides, from about 15 nucleotides to about 30 nucleotides, and from about 20 nucleotides to about 25 nucleotides. Oligonucleotides may be chemically synthesized and can also be used as probes. In some embodiments oligonucleotides are single stranded. In some embodiments oligonucleotides comprise at least one portion which is double stranded. In some embodiments the oligonucleotides are antisense oligonucleotides (ASO). In some embodiments the oligonucleotides are RNA interference oligonucleotides (RNAi oligonucleotides).

As used herein, the term “antisense oligonucleotide” refers to an unmodified or modified nucleic acid having a nucleotide sequence complementary to an FGF21 polynucleotide sequence including polynucleotide sequences associated with the transcription or translation of FGF21 (e.g., a promoter of an FGF21 polynucleotide), where the antisense polynucleotide is capable of hybridizing to an FGF21 polynucleotide sequence. Of particular interest are antisense polynucleotides capable of inhibiting transcription and/or translation of FGF21 polypeptide-encoding polynucleotide either in vitro or in vivo.

As used herein, the terms “siRNA oligonucleotides”, “RNAi oligonucleotides”, “short interfering RNA”, or “siRNA” are used interchangeably and refer to oligonucleotides that work through post-transcriptional gene silencing, also known as RNA interference (RNAi). The terms refer to a double stranded nucleic acid molecule capable of RNA interference “RNAi”, (see Kreutzer et al., WO 00/44895; Zernicka-Goetz et al. WO 01/36646; Fire, WO 99/32619; Mello and Fire, WO 01/29058). SiRNA molecules are generally RNA molecules but further encompass chemically modified nucleotides and non-nucleotides. SiRNA gene-targeting experiments have been carried out by transient siRNA transfer into cells (achieved by such classic methods as liposome-mediated transfection, electroporation, or microinjection). Molecules of siRNA are 21- to 23-nucleotide RNAs, with characteristic 2- to 3-nucleotide 3′-overhanging ends resembling the RNase III processing products of long double-stranded RNAs (dsRNAs) that normally initiate RNAi.

As used herein, a “target region” of an FGF21 RNA, is a region to which an oligonucleotide hybridizes, such as an ASO or siRNA. The target region can be perfectly complementary with an ASO or antisense strand or a siRNA, or there may be at least one, two, three, or four mismatches between the target region and the ASO or siRNA.

As used herein, the term “decoy” refers to a polypeptide comprising at least a portion of an FGF21 polypeptide capable of binding an FGF21 receptor, such as FGFR-1 or FGFR-2. In some embodiments the decoy is capable of binding a phosphorylated FGF21 receptor.

As used herein, the term “single nucleotide polymorphism” (“SNP”) refers to a nucleotide substitution, nucleotide insertion or nucleotide deletion, which in the case of insertion and deletion, includes insertion or deletion of one or more nucleotides at a position of a gene.

As used herein, the term “therapeutically effective amount” is meant to refer to an amount of a medicament which produces a medicinal effect observed as reduction or reverse in one or more clinical endpoints, growth and/or survival of cancer cell, or metastasis of cancer cells in an individual when a therapeutically effective amount of the medicament is administered to the individual. Therapeutically effective amounts are typically determined by the effect they have compared to the effect observed when a composition which includes no active ingredient is administered to a similarly situated individual. The precise effective amount for a subject will depend upon the subject's size and health, the nature and extent of the condition, and the therapeutics or combination of therapeutics selected for administration. However, the effective amount for a given situation is determined by routine experimentation and is within the judgment of the clinician.

As used herein, the terms “in combination with” or “in conjunction with” refer to administration of the FGF21 modulators of the invention with other therapeutic regimens.

As used herein, the term “susceptible” refers to patients for whom FGF21 therapy is an acceptable method of treatment, i.e., patients who are likely to respond positively. Cancer patients susceptible to FGF21 therapy express high levels of FGF21 relative to those patients not susceptible to FGF21 therapy. Cancer patients who are not good candidates for FGF21 therapy include cancer patients with tumor samples that lack or have lower levels of FGF21 in or on their cancer cells.

As used herein, the term “detecting” means to establish, discover, or ascertain evidence of an activity (for example, gene expression) or biomolecule (for example, a polypeptide).

A “native sequence” polypeptide is one that has the same amino acid sequence as a polypeptide derived from nature. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “amino acid sequence variant” refers to polypeptides having amino acid sequences that differ to some extent from a native sequence polypeptide. Ordinarily, amino acid sequence variants will possess at least about 85%, at least about 90%, at least about 95%, at least about 98% or at least about 99% homology with at least one receptor binding domain of a native ligand or with at least one ligand binding domain of a native receptor. The amino acid sequence variants possess substitutions, deletions, and/or insertions at certain positions within the amino acid sequence of the native amino acid sequence.

As used herein, the phrase “homologous nucleotide sequence,” or “homologous amino acid sequence,” or variations thereof, refers to sequences characterized by a homology, at the nucleotide level or amino acid level, of at least a specified percentage and is used interchangeably with “sequence identity”. Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals. Homologous nucleotide sequences also include, but are not limited to, naturally occurring allelic variations and mutations of the nucleotide sequences set forth herein. In some embodiments homologous nucleotide sequences encode polypeptides with the same or similar binding characteristics and/or activity as the wild-type sequence. Homologous amino acid sequences include those amino acid sequences which contain conservative amino acid substitutions and which polypeptides have the same or similar binding characteristics and/or activity as the wild-type sequence. In some embodiments, a nucleotide or amino acid sequence is homologous if it has at least 70%, 80%, 85%, 90%, 95%, 96%, 97%, 98%, or 99% identity. In some embodiments, a nucleotide or amino acid sequence is homologous if it has 1-10, 10-20, 20-30, 30-40, 40-50, or 50-60 nucleotide/amino acid substitutions, additions, or deletions. In some embodiments, the homologous amino acid sequences have no more than 5 or no more than 3 conservative amino acid substitutes.

Percent homology or identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, homology between the probe and target is between about 75% to about 85%. In some embodiments, nucleic acids have nucleotides that are at least about 85%, about 90%, about 92%, about 94%, about 95%, about 97%, about 98%, about 99% and about 100% homologous to SEQ ID NO:1, or a portion thereof. Complements of such sequences are also provided. In some embodiments, the complement is a full and complete complement of the nucleotide sequence.

Homology may also be at the polypeptide level. In some embodiments, polypeptides are about 85%, about 90%, about 92%, about 94%, about 95%, about 97%, about 98%, about 99% and about 100% homologous to SEQ ID NO:2, or a portion thereof. In some embodiments the polypeptides have up to 5, up to 10, up to 15, up to 20 or up to 30 amino acid insertions, deletions or substitutions.

As used herein, the term “probe” refers to nucleic acid sequences of variable length. In some embodiments probes comprise at least about 10 and as many as about 6,000 nucleotides. In some embodiments probes comprise at least 12, at least 14, at least 16, at least 18, at least 20, at least 25, at least 50 or at least 75 consecutive nucleotides. Probes are used in the detection of identical, similar, or complementary nucleic acid sequences. Longer length probes are usually obtained from natural or recombinant sources, are highly specific to the target sequence, and are much slower to hybridize to the target than are oligomers. Probes may be single- or double-stranded and are designed to have specificity in PCR, hybridization membrane-based, in situ hybridization (ISH), fluorescent in situ hybridization (FISH), or ELISA-like technologies.

As used herein, the term “mixing” refers to the process of combining one or more compounds, cells, molecules, and the like together in the same area. This may be performed, for example, in a test tube, petri dish, or any container that allows the one or more compounds, cells, or molecules, to be mixed.

As used herein the term “isolated” refers to a polynucleotide, a polypeptide, an antibody, or a host cell that is in an environment different from that in which the polynucleotide, the polypeptide, or the antibody naturally occurs. Methods of isolating cells are well known to those skilled in the art. A polynucleotide, a polypeptide, or an antibody which is isolated is generally substantially purified.

As used herein, the term “substantially purified” refers to a compound (e.g., either a polynucleotide or a polypeptide or an antibody) that is removed from its natural environment and is at least 60% free, at least 75% free, and at least 90% free from other components with which it is naturally associated.

As used herein, the term “binding” means the physical or chemical interaction between two or more biomolecules or compounds. Binding includes ionic, non-ionic, hydrogen bonds, Van der Waals, hydrophobic interactions, etc. Binding can be either direct or indirect; indirect being through or due to the effects of another biomolecule or compound. Direct binding refers to interactions that do not take place through or due to the effect of another molecule or compound but instead are without other substantial chemical intermediates.

As used herein, the term “contacting” means bringing together, either directly or indirectly, one molecule into physical proximity to a second molecule. The molecule can be in any number of buffers, salts, solutions, etc. “Contacting” includes, for example, placing a polynucleotide into a beaker, microtiter plate, cell culture flask, or a microarray, or the like, which contains a nucleic acid molecule. Contacting also includes, for example, placing an antibody into a beaker, microtiter plate, cell culture flask, or microarray, or the like, which contains a polypeptide. Contacting may take place in vivo, ex vivo, or in vitro.

As used herein, the phrase “stringent hybridization conditions” or “stringent conditions” refers to conditions under which a probe, primer, or oligonucleotide will hybridize to its target sequence, but to a minimal number of other sequences. Stringent conditions are sequence-dependent and will be different in different circumstances. Longer sequences will hybridize with specificity to their proper complements at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tₘ, 50% of the probes are hybridized to their complements at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 to 50 nucleotides) and at least about 60° C. for longer probes, primers or oligonucleotides. Stringent conditions may also be achieved with the addition of destabilizing agents, such as formamide.

As used herein, the term “moderate stringency conditions” refers to conditions under which a probe, primer, or oligonucleotide will hybridize to its target sequence, but to a limited number of other sequences. Moderate conditions are sequence-dependent and will be different in different circumstances. Moderate conditions are well-known to the art skilled and are described in, inter alis, Maniatis et al. (Molecular Cloning: A Laboratory Manual, Cold Spring Harbor Laboratory; 2nd Edition (December 1989)).

The nucleic acid compositions described herein can be used, for example, to produce polypeptides, as probes for the detection of mRNA in biological samples (e.g., extracts of human cells) or cDNA produced from such samples, to generate additional copies of the polynucleotides, to generate ribozymes or oligonucleotides (single and double stranded), and as single stranded DNA probes or as triple-strand forming oligonucleotides. The probes described herein can be used to, for example, determine the presence or absence of the polynucleotides provided herein in a sample. The polypeptides can be used to generate antibodies specific for a polypeptide associated with cancer, which antibodies are in turn useful in diagnostic methods, prognostic methods, and the like as discussed in more detail herein. Polypeptides are also useful as targets for therapeutic intervention, as discussed in more detail herein. Antibodies of the present invention may also be used, for example, to purify, detect, and target the polypeptides of the present invention, including both in vitro and in vivo diagnostic and therapeutic methods. For example, the antibodies are useful in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988). These and other uses are described in more detail below.

As used herein the term “imaging agent” refers to a composition linked to an antibody, small molecule, or probe of the invention that can be detected using techniques known to the art-skilled. As used herein, the term “evidence of gene expression” refers to any measurable indicia that a gene is expressed.

The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Specific examples of cancers that can be treated by the methods and compositions of the present invention include, but are not limited to, FGF21-associated cancers. As used herein, “FGF21-associated cancer” refers to a cancer characterized by cells that differentially express FGF21 relative to non-cancerous cells. The present invention is also applicable to any tumor cell-type where FGF21 plays a role in cancer cell growth, tumor formation, cancer cell proliferation, cancer cell metastasis, cell migration, angiogenesis, FGF21 signaling, FGF21-mediated cell-cell adhesion, cell-cell interaction, and FGF21 expression. In some embodiments, the cancer is colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma and melanoma. In some embodiments, the cancer is colon cancer. In some embodiments, such cancers exhibit differential expression of FGF21 of at least about 25%, at least about 50%, at least about 75%, at least about 100%, at least about 150%, at least about 200%, or at least about 300% as compared to a control.

The present invention provides methods and compositions that provide for the treatment, inhibition, and management of diseases and disorders associated with FGF21 expression as well as the treatment, inhibition, and management of symptoms of such diseases and disorders. Some embodiments of the invention relate to methods and compositions that treat, inhibit or manage diseases or disorders associated with FGF21 overexpression. For example, some embodiments of the invention relate to methods and compositions comprising compositions that treat, inhibit or manage cancer including, without limitation, cancer metastases, cancer cell proliferation, cancer cell growth and cancer cell invasion. Some embodiments of the invention relate to methods and compositions comprising compositions that treat, inhibit or manage diseases or disorders that benefit from FGF21 expression or overexpression. For example, some embodiments of the invention relate to methods and compositions that treat, inhibit or manage vascular diseases, including, without limitation, CAD and PAD.

Treatment of Cancers

For the treatment of diseases or disorders associated with overexpression of FGF21, such as a cancer, the present invention provides methods including other active ingredients in combination with the FGF21 modulators of the present invention. In some embodiments, the methods further comprise administering one or more conventional cancer therapeutics to the patient. In some embodiments the methods of the present invention further comprise treating the patient with one or more of chemotherapy, radiation therapy, hormone ablation, or surgery.

The present invention also provides methods and compositions for the treatment, inhibition, and management of cancer or other hyperproliferative cell disorder or diseases that has become partially or completely refractory to current or standard cancer treatment, such as surgery, chemotherapy, radiation therapy, hormonal therapy, and biological therapy.

The invention also provides diagnostic and/or imaging methods using the FGF21 modulators of the invention, particularly FGF21 antibodies, to diagnose cancer and/or predict cancer progression. In some embodiments, the methods of the invention provide methods of imaging and localizing tumors and/or metastases and methods of diagnosis and prognosis. In some embodiments, the methods of the invention provide methods to evaluate the appropriateness of FGF21-related therapy.

Treatment of Vascular Disease

For the treatment of diseases or disorders that benefit from FGF21 expression or overexpression, such as vascular disease, the present invention provides methods including other active ingredients in combination with the FGF21 modulators of the present invention. In some embodiments, the methods further comprise administering one or more conventional cancer therapeutics to the patient. In some embodiments, the methods of the present invention further comprise angioplasty, stenting, atherectomy, bypass surgery, or administration of medications such as antiplatelet agents, anticoagulants, or thrombolytics. In some embodiments the methods of the present invention further comprise putting the patient on a special diet to control fat and cholesterol intake, or on a special exercise regimen.

The present invention also provides methods and compositions for the treatment, inhibition, and management of a vascular disorder or disease that has become partially or completely refractory to current or standard treatments.

The invention also provides diagnostic and/or imaging methods using the FGF21 modulators of the invention, particularly FGF21 antibodies, to diagnose vascular disease and/or assess disease progression. In some embodiments, the methods of the invention provide methods of imaging new blood vessel growth such as to monitor the progress of a treatment regimen. In some embodiments, the methods of the invention provide methods to evaluate the appropriateness of FGF21-related therapy.

FGF21 Modulators

The present invention provides FGF21 modulators for, inter alia, the treatment, diagnosis, detection or imaging of cancer, or for the treatment, diagnosis, detection or imaging of vascular disease. FGF21 modulators are also useful in the preparation of medicaments for the treatment of cancer and vascular disease.

In some embodiments, the FGF21 modulator is an oligonucleotide, a small molecule, a mimetic, a decoy, or an antibody. In some embodiments, the FGF21 modulator inhibits an FGF21 biological activity by 25%, 50%, 75%, 80%, 90%, 95%, 97%, 98%, 99% or 100%, as compared to a control. In some embodiments, the FGF21 modulator inhibits FGF21 expression by at least 25%, 50%, 75%, 80%, 90%, 95%, 97%, 98%, 99% or 100%, as compared to a control. In some embodiments, the FGF21 modulator upregulates an FGF21 biological activity by at least 25%, 50%, 75%, 100%, 150%, 200%, 250%, 400% or 500% as compared to a control. In some embodiments, the FGF21 modulator upregulates FGF21 expression by at least 25%, 50%, 75%, 100%, 150%, 200%, 250%, 400% or 500% as compared to a control.

In some embodiments, the FGF21 activity modulated is one or more of cell proliferation (e.g., endothelial cell proliferation), angiogenesis, blood vessel formation, cell signaling, kinase activity, glucose uptake by adipocytes, cancer cell survival, interactions between FGF21 and an FGF21 receptor, phosphorylation of an FGF21 receptor, or apoptosis. The FGF21 receptor can be, for example, FGFR-1 or FGFR-2. Phosphorylation of an FGF21 receptor can be tyrosine phosphorylation of an FGF21 receptor.

In some embodiments, FGF21 modulators comprise or are directed to antigenic regions of the FGF21 polypeptide. Antigenic regions of FGF21 include, without limitation:

(SEQ ID NO: 204)
RQRYLYTDDAQQTEAHLEI;
(SEQ ID NO: 205)
GAADQSPESLLQLKALKPGV;
(SEQ ID NO: 206)
FLCQRPDGALYGSLHFDPE;
(SEQ ID NO: 207)
QSEAHGLPLHLPGNKSPHRDPAPRGPARFLPLPGL;
and
(SEQ ID NO: 208)
MVGPSQGRSPSYAS

Antibodies

In some embodiments the FGF21 modulator is a monoclonal antibody, a polyclonal antibody, a chimeric antibody, a human antibody, a humanized antibody, a single-chain antibody, or a Fab fragment. The antibody may be labeled with, for example, an enzyme, radioisotope, or fluorophore. In some embodiments the antibody has a binding affinity less than about 1×10⁵ Ka for a polypeptide other than FGF21. In some embodiments, the FGF21 modulator is a monoclonal antibody which binds to FGF21 with an affinity of at least 1×10⁸ Ka.

The invention also provides antibodies that competitively inhibit binding of an antibody to an epitope of the invention as determined by any method known in the art for determining competitive binding using, for example, immunoassays. In some embodiments, the antibody competitively inhibits binding to the epitope by at least 95%, at least 90%, at least 85%, at least 80%, at least 75%, at least 70%, at least 60%, or at least 50%.

In some embodiments the antibody is a humanized antibody. Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); Jones et al., Nature 321:522-525 (1986); Morrison et al., Proc. Natl. Acad. Sci, U.S.A., 81:6851-6855 (1984); Morrison and 01, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

Antibodies of the present invention may function through different mechanisms. In some embodiments, antibodies trigger antibody-dependent cellular cytotoxicity (ADCC), a lytic attack on antibody-targeted cells. In some embodiments, antibodies have multiple therapeutic functions, including, for example, antigen-binding, induction of apoptosis, and complement-dependent cellular cytotoxicity (CDC).

In some embodiments, antibodies of the present invention may act as agonists or antagonists of the polypeptides of the present invention. For example, in some embodiments the present invention provides antibodies which disrupt the receptor/ligand interactions with the polypeptides of the invention either partially or fully. In some embodiments antibodies of the present invention bind an epitope disclosed herein, or a portion thereof. In some embodiments, antibodies are provided that modulate ligand activity or receptor activity by at least 95%, at least 90%, at least 85%, at least 80%, at least 75%, or at least 50% compared to the activity in the absence of the antibody.

In some embodiments, FGF21 antibodies inhibit the interaction of FGF21 with an FGF21 receptor, such as FGFR-1 or FGFR-2, or inhibit phosphorylation of an FGF21 receptor. In some embodiments, FGF21 antibodies inhibit angiogenesis, cell proliferation, cell signaling, kinase activity (such as tyrosine kinase activity), glucose uptake into adipocytes, and cancer cell survival. In some embodiments, FGF21 antibodies increase apoptosis. In some embodiments, FGF21 antibodies cause apoptosis in at least 30% of cells in a cell population as compared to a control cell population.

In some embodiments the present invention provides neutralizing antibodies. In some embodiments the neutralizing antibodies act as receptor antagonists, i.e., inhibiting either all or a subset of the biological activities of the ligand-mediated receptor activation. In some embodiments the antibodies may be specified as agonists, antagonists or inverse agonists for biological activities comprising the specific biological activities of the peptides of the invention disclosed herein.

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

In addition to chimeric and humanized antibodies, fully human antibodies can be derived from transgenic mice having human immunoglobulin genes (see, e.g., U.S. Pat. Nos. 6,075,181, 6,091,001, and 6,114,598, all of which are incorporated herein by reference), or from phage display libraries of human immunoglobulin genes (see, e.g. McCafferty et al., Nature, 348:552-554 (1990). Clackson et al., Nature, 352:624-628 (1991), and Marks et al., J. Mol. Biol., 222:581-597 (1991)). In some embodiments, antibodies may be produced and identified by scFv-phage display libraries. Antibody phage display technology is available from commercial sources such as from Xoma (Berkeley, Calif.).

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution in saline, preferably in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial or limiting dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

As an alternative to the use of hybridomas for expression, antibodies can be produced in a cell line such as a CHO or myeloma cell line, as disclosed in U.S. Pat. Nos. 5,545,403; 5,545,405; and 5,998,144; each incorporated herein by reference. Briefly the cell line is transfected with vectors capable of expressing a light chain and a heavy chain, respectively. By transfecting the two proteins on separate vectors, chimeric antibodies can be produced. Immunol. 147:8; Banchereau et al. (1991) Clin. Immunol. Spectrum 3:8; and Banchereau et al. (1991) Science 251:70; all of which are herein incorporated by reference.

Human antibodies can also be produced using techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1): δ 95 (1991)). Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779 783 (1992); Lonberg et al., Nature 368 856 859 (1994); Morrison, Nature 368, 812 13 (1994); Fishwild et al., Nature Biotechnology 14, 845 51 (1996); Neuberger, Nature Biotechnology 14, 826 (1996); Lonberg and Huszar, Intern. Rev. Immunol. 13 65 93 (1995); Jones et al., Nature 321:522-525 (1986); Morrison et al., Proc. Natl. Acad. Sci, U.S.A., 81:6851-6855 (1984); Morrison and 01, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference. Fully humanized antibodies can be identified in screening assays using commercial resources such as Morphosys (Martinsried/Planegg, Germany).

Humanized antibodies can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Antibodies of the present invention can also be produced using human engineering techniques as discussed in U.S. Pat. No. 5,766,886, which is incorporated herein by reference.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

Antibodies of the present invention may be administered to a subject via in vivo therapeutic antibody gene transfer as discussed by Fang et al. (2005), Nat. Biotechnol. 23, 584-590. For example recombinant vectors can be generated to deliver a multicistronic expression cassette comprising a peptide that mediates enzyme independent, cotranslational self cleavage of polypeptides placed between MAb heavy and light chain encoding sequences. Expression leads to stoichiometric amounts of both MAb chains. A preferred example of the peptide that mediates enzyme independent, cotranslational self cleavage is the foot-and-mouth-disease derived 2A peptide.

In some embodiments, fragments of the antibodies retain the desired affinity of the full-length antibody. Thus, in some embodiments, a fragment of an anti-FGF21 antibody will retain the ability to bind to FGF21. Such fragments are characterized by properties similar to the corresponding full-length anti-FGF21 antibody, that is, the fragments will specifically bind a human FGF21 antigen expressed on the surface of a human cell.

In some embodiments, the antibodies bind to one or more epitopes in an extracellular domain of FGF21. In some embodiments, the antibodies modulate one or more FGF21-related biological activities. In some embodiments the antibodies inhibit one or more of cancer cell growth, tumor formation, and cancer cell proliferation.

In some embodiments, the antibody is a monoclonal antibody which binds to one or more FGF21 epitopes in a domain selected from the group consisting of the N-terminal signal peptide domain of FGF21, or the receptor binding domain of FGF21.

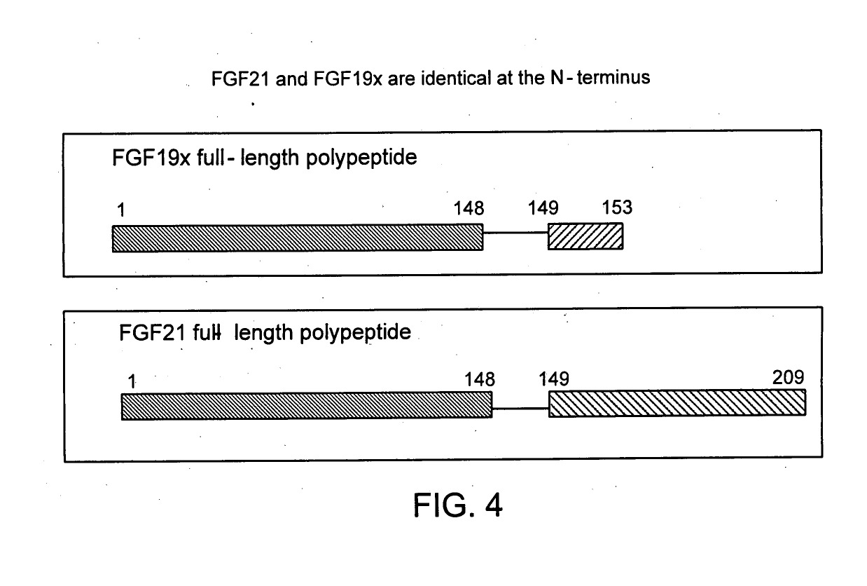

In some embodiments, the monoclonal antibody binds to an FGF21 epitope in the C-terminal domain of FGF21. In some embodiments, the monoclonal antibody binds to an FGF21 epitope in region 2 or region 5 of FGF21 as indicated in Example 4 (see also FIG. 4).

Suitable antibodies according to the present invention can recognize linear or conformational epitopes, or combinations thereof. In some embodiments the antibodies of the present invention bind to epitopes of antigenic regions of FGF21 selected from the group consisting of SEQ ID NOs:3-203. In some embodiments the antibody is specific for an epitope having a sequence selected from the group consisting of SEQ ID NOs:117-203. In some embodiments the antibody is specific for an epitope having a sequence selected from the group consisting of SEQ ID NOS:186-203. It is to be understood that these peptides may not necessarily precisely map one epitope, but may also contain FGF21 sequence that is not immunogenic.

Methods of predicting other potential epitopes to which an antibody of the invention can bind are well-known to those of skill in the art and include without limitation, Kyte-Doolittle Analysis (Kyte, J. and Dolittle, R. F., J. Mol. Biol. (1982) 157:105-132), Hopp and Woods Analysis (Hopp, T. P. and Woods, K. R., Proc. Natl. Acad. Sci. USA (1981) 78:3824-3828; Hopp, T. J. and Woods, K. R., Mol. Immunol. (1983) 20:483-489; Hopp, T. J., J. Immunol. Methods (1986) 88:1-18.), Jameson-Wolf Analysis (Jameson, B. A. and Wolf, H., Comput. Appl. Biosci. (1988) 4:181-186.), and Emini Analysis (Emini, E. A., Schlief, W. A., Colonno, R. J. and Wimmer, E., Virology (1985) 140:13-20.).

In some embodiments, potential epitopes are identified by determining theoretical extracellular domains. Analysis algorithms such as TMpred (see K. Hofmann & W. Stoffel (1993) TMbase—A database of membrane spanning proteins segments Biol. Chem. Hoppe-Seyler 374,166) or TMHMM (Krogh et al., Predicting transmembrane protein topology with a hidden Markov model: Application to complete genomes. Journal of Molecular Biology, 305:567-580, 2001) can be used to make such predictions. Other algorithms, such as SignalP 3.0 (Bednsten et al., J Mol. Biol. 340:783-95, 2004) can be used to predict the presence of signal peptides and to predict where those peptides would be cleaved from the full-length protein. The portions of the proteins on the outside of the cell can serve as targets for antibody interaction.

Antibodies are defined to be “specifically binding” if: 1) they exhibit a threshold level of binding activity, and/or 2) they do not significantly cross-react with known related polypeptide molecules. The binding affinity of an antibody can be readily determined by one of ordinary skill in the art, for example, by Scatchard analysis (Scatchard, Ann. NY Acad. Sci. 51: 660-672, 1949). In some embodiments the antibodies of the present invention bind to their target epitopes or mimetic decoys at least 1.5-fold, 2-fold, 5-fold 10-fold, 100-fold, 10³-fold, 10⁴-fold, 10⁵-fold, 10⁶-fold or greater for the target cancer-associated polypeptide.

In some embodiments the antibodies bind with high affinity of 10⁻⁴M or less, 10⁻⁷M or less, 10⁻⁹M or less or with subnanomolar affinity (0.9, 0.8, 0.7, 0.6, 0.5, 0.4, 0.3, 0.2, 0.1 nM or even less). In some embodiments the binding affinity of the antibodies for FGF21 is at least 1×10⁶ Ka. In some embodiments the binding affinity of the antibodies for FGF21 is at least 5×10⁶ Ka, at least 1×10⁷ Ka, at least 2×10⁷ Ka, at least 1×10⁸ Ka, or greater. Antibodies of the present invention may also be described or specified in terms of their binding affinity to a polypeptide of the invention. In some embodiments binding affinities include those with a Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶M, 5×10⁻⁷ M, 10⁻⁷ M, 5×10⁻⁸ M, 10⁻⁸ M, 5×10⁻⁹ M, 10⁻⁹M, 5×10⁻¹⁰ M, 10⁻¹⁰ M, 5×10⁻¹¹ M, 10⁻¹¹ M, 5×10⁻¹² M, 10⁻¹² M, 5×10⁻¹³M, 10⁻¹³ M, 5×10⁻¹⁴M, 10⁻¹⁴M, 5×10⁻¹⁵M, or 10⁻¹⁵M, or less.

In some embodiments, the antibodies of the present invention bind FGF21 polypeptide but not known related family members of FGF21. Binding of an antibody to FGF21 and related polypeptides can be assayed using standard Western blot analysis (Ausubel et al.). Examples of known related polypeptides include, without limitation, other members of the FGF protein family (e.g., FGF19x (WO 01/18209), and the like).

In some embodiments, the antibodies of the present invention bind to orthologs, homologs, paralogs or variants, or combinations and subcombinations thereof, of FGF21. In some embodiments, the antibodies of the present invention bind to orthologs of FGF21. In some embodiments, the antibodies of the present invention bind to homologs of FGF21. In some embodiments, the antibodies of the present invention bind to paralogs of FGF21. In some embodiments, the antibodies of the present invention bind to variants of FGF21. In some embodiments, the antibodies of the present invention do not bind to orthologs, homologs, paralogs or variants, or combinations and subcombinations thereof, of FGF21. In some embodiments, the antibodies of the present invention do not specifically bind to FGF19x.

In some embodiments, antibodies may be screened against known related polypeptides to isolate an antibody population that specifically binds to FGF21 polypeptides. For example, antibodies specific to human FGF21 polypeptides will flow through a column comprising other FGF proteins (FGF19x, and the like) adhered to insoluble matrix under appropriate buffer conditions. Such screening allows isolation of polyclonal and monoclonal antibodies non-crossreactive to closely related polypeptides (Antibodies: A Laboratory Manual, Harlow and Lane (eds.), Cold Spring Harbor Laboratory Press, 1988; Current Protocols in Immunology, Cooligan et al. (eds.), National Institutes of Health, John Wiley and Sons, Inc., 1995). Screening and isolation of specific antibodies is well known in the art (see, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984). Representative examples of such assays include: concurrent immunoelectrophoresis, radioimmunoassay (RIA), radioimmunoprecipitation, enzyme-linked immunosorbent assay (ELISA), dot blot or Western blot assay, inhibition or competition assay, and sandwich assay.

In some embodiments, the antibodies of the present invention do not specifically bind to epitopes consisting of a sequence selected from the group consisting of SEQ ID NOs:3-111, SEQ ID NOs:133-138, or SEQ ID NOs:160-164 (Table 2). In some embodiments, the antibodies of the present invention do not specifically bind to epitopes consisting of residues 1-49 of SEQ ID NO:2. In some embodiments, the antibodies do not bind FGF19x.

The invention also provides antibodies that are SMIPs or binding domain immunoglobulin fusion proteins specific for target protein. These constructs are single-chain polypeptides comprising antigen binding domains fused to immunoglobulin domains necessary to carry out antibody effector functions. See e.g., WO03/041600, U.S. Patent Publication 20030133939 and U.S. Patent Publication 20030118592.

In some embodiments the antibodies of the present invention are neutralizing antibodies. A neutralizing antibody binds an infectious agent, such as a virus or a bacterium, such as a virus or bacterium associated with cancer (e.g., a JC polyoma virus, Epstein-Barr virus, or Helicobacter pylori). In some embodiments the neutralizing antibodies can effectively act as receptor antagonists, i.e., inhibiting either all or a subset of the biological activities of the ligand-mediated receptor activation. In some embodiments the antibodies may be specified as agonists, antagonists or inverse agonists for biological activities comprising the specific biological activities of the peptides of the invention disclosed herein.

The antibodies of the present invention can be screened for the ability to either be rapidly internalized upon binding to the tumor-cell antigen in question, or for the ability to remain on the cell surface following binding. In some embodiments, for example in the construction of some types of immunoconjugates, the ability of an antibody to be internalized may be desired if internalization is required to release the toxin moiety. Alternatively, if the antibody is being used to promote ADCC or CDC, it may be more desirable for the antibody to remain on the cell surface. A screening method can be used to differentiate these type behaviors. For example, a tumor cell antigen bearing cell may be used where the cells are incubated with human IgG1 (control antibody) or one of the antibodies of the invention at a concentration of approximately 1 μg/mL on ice (with 0.1% sodium azide to block internalization) or 37° C. (without sodium azide) for 3 hours. The cells are then washed with cold staining buffer (PBS+1% BSA+0.1% sodium azide), and are stained with goat anti-human IgG-FITC for 30 minutes on ice. Geometric mean fluorescent intensity (MFI) is recorded by FACS Calibur. If no difference in MFI is observed between cells incubated with the antibody of the invention on ice in the presence of sodium azide and cells observed at 37° C. in the absence of sodium azide, the antibody will be suspected to be one that remains bound to the cell surface, rather than being internalized. If however, a decrease in surface stainable antibody is found when the cells are incubated at 37° C. in the absence of sodium azide, the antibody will be suspected to be one which is capable of internalization.

Antibody Conjugates

In some embodiments, the antibodies of the invention are conjugated. In some embodiments, the conjugated antibodies are useful for cancer therapeutics, cancer diagnosis, or imaging of cancerous cells. In some embodiments, the conjugated antibodies are useful for diagnosis or imaging of vascular disease or disorder.

For diagnostic applications, the antibody typically will be labeled with a detectable moiety. Numerous labels are available which can be generally grouped into the following categories:

(a) Radionuclides such as those discussed infra. The antibody can be labeled, for example, with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

The antibodies may also be used for in vivo diagnostic assays. In some embodiments, the antibody is labeled with a radionuclide so that the tumor can be localized using immunoscintiography. As a matter of convenience, the antibodies of the present invention can be provided in a kit, i.e., a packaged combination of reagents in predetermined amounts with instructions for performing the diagnostic assay. Where the antibody is labeled with an enzyme, the kit may include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In some embodiments, antibodies are conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52: 127-131 (1992)) to generate a maytansinoid-antibody immunoconjugate. In some embodiments, the conjugate may be the highly potent maytansine derivative DM1 (N2′-deacetyl-N2′-(3-mercapto-1-oxopropyl)-maytansine) (see for example WO02/098883 published Dec. 12, 2002) which has an IC50 of approximately 10-11 M (review, see Payne (2003) Cancer Cell 3:207-212) or DM4 (N2′-deacetyl-N2′(4-methyl-4-mercapto-1-oxopentyl)-maytansine) (see for example WO2004/103272 published Dec. 2, 2004).

In some embodiments the antibody conjugate comprises an anti-tumor cell antigen antibody conjugated to one or more calicheamicin molecules. The calicheamicin family of antibiotics is capable of producing double-stranded DNA breaks at sub-picomolar concentrations. Structural analogues of calicheamicin which may be used include, but are not limited to, gamma1I, alpha2I, alpha3I, N-acetyl-gamma1I, PSAG and thetaI1 (Hinman et al. Cancer Research 53: 3336-3342 (1993) and Lode et al. Cancer Research 58: 2925-2928 (1998)). See, also, U.S. Pat. Nos. 5,714,586; 5,712,374; 5,264,586; and 5,773,001, each of which is expressly incorporated herein by reference.

In some embodiments the antibody is conjugated to a prodrug capable of being released in its active form by enzymes overproduced in many cancers. For example, antibody conjugates can be made with a prodrug form of doxorubicin wherein the active component is released from the conjugate by plasmin. Plasmin is known to be over produced in many cancerous tissues (see Decy et al, (2004) FASEB Journal 18(3): 565-567).

In some embodiments the antibodies are conjugated to enzymatically active toxins and fragments thereof. In some embodiments the toxins include, without limitation, diphtheria A chain, nonbinding active fragments of diphtheria toxin, exotoxin A chain (from Pseudomonas aeruginosa), Pseudomonas endotoxin, ricin A chain, abrin A chain, modeccin A chain, alpha-sarcin, Aleurites fordii proteins, dianthin proteins, Phytolaca americana proteins (PAPI, PAPII, and PAP-S), Ribonuclease (RNase), Deoxyribonuclease (DNase), pokeweed antiviral protein, momordica charantia inhibitor, curcin, crotin, sapaonaria officinalis inhibitor, gelonin, mitogellin, restrictocin, phenomycin, neomycin and the tricothecenes. See, for example, WO 93/21232 published Oct. 28, 1993. In some embodiments the toxins have low intrinsic immunogenicity and a mechanism of action (e.g. a cytotoxic mechanism versus a cytostatic mechanism) that reduces the opportunity for the cancerous cells to become resistant to the toxin.

In some embodiments conjugates are made between the antibodies of the invention and immunomodulators. For example, in some embodiments immunostimulatory oligonucleotides can be used. These molecules are potent immunogens that can elicit antigen-specific antibody responses (see Datta et al, (2003) Ann N.Y. Acad. Sci. 1002: 105-111). Additional immunomodulatory compounds can include stem cell growth factor such as “Si factor”, lymphotoxins such as tumor necrosis factor (TNF), hematopoietic factor such as an interleukin, colony stimulating factor (CSF) such as granulocyte-colony stimulating factor (G-CSF) or granulocyte macrophage-stimulating factor (GM-CSF), interferon (IFN) such as interferon alpha, beta or gamma, erythropoietin, and thrombopoietin.

In some embodiments radioconjugated antibodies are provided. In some embodiments such antibodies can be made using ³²P, ³³P, ⁴⁷Sc, ⁵⁹Fe, ⁶⁴Cu, ⁶⁷Cu, ⁷⁵Se, ⁷⁷As, ⁸⁹Sr, ⁹⁰Y, ⁹⁹Mo, ¹⁰⁵Rh, ¹⁰⁹Pd, ¹²⁵I, ¹³¹I, ¹⁴²Pr, ¹⁴³Pr, ¹⁴⁹Pm, ¹⁵³Sm, ¹⁶¹Th, ¹⁶⁶Ho, ¹⁶⁹Er, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹Pb, ²¹²Pb, ²¹³Bi, ⁵⁸Co, ⁶⁷Ga, ^{80m}Br, ^{99m}Tc, ^{103m}Rh, ¹⁰⁹Pt, ¹⁶¹Ho, ^{189m}Os, ¹⁹²Ir, ¹⁵²Dy, ²¹¹At, ²¹²Bi, ²²³Ra, ²¹⁹Rn, ²¹⁵Po, ²¹¹Bi, ²²⁵Ac, ²²¹Fr, ²¹⁷At, ²¹³Bi, ²⁵⁵Fm and combinations and subcombinations thereof. In some embodiments, boron, gadolinium or uranium atoms are conjugated to the antibodies. In some embodiments the boron atom is ¹⁰B, the gadolinium atom is ¹⁵⁷Gd and the uranium atom is ²³⁵U.

In some embodiments the radionuclide conjugate has a radionuclide with an energy between 20 and 10,000 keV. The radionuclide can be an Auger emitter, with an energy of less than 1000 keV, a P emitter with an energy between 20 and 5000 keV, or an alpha or ‘α’ emitter with an energy between 2000 and 10,000 keV.

In some embodiments diagnostic radioconjugates are provided which comprise a radionuclide that is a gamma-, beta-, or positron-emitting isotope. In some embodiments the radionuclide has an energy between 20 and 10,000 keV. In some embodiments the radionuclide is selected from the group of ¹⁸F, ⁵¹Mn, ^{52m}Mn, ⁵²Fe, ⁵⁵Co, ⁶²Cu, ⁶⁴Cu, ⁶⁸Ga, ⁷²As, ⁷⁵Br, ⁷⁶Br, ^{82m}Rb, ⁸³Sr, ⁸⁹Zr, ^{94m}Tc, ⁵¹Cr, ⁵⁷Co, ⁵⁸Co, ⁵⁹Fe, ⁶⁷Ga, ⁷⁵Se, ⁹⁷Ru, ^{99m}Tc, ^{114m}In, ¹²³I, ¹²⁵I, ¹³Li and ¹⁹⁷Hg.

In some embodiments the antibodies of the invention are conjugated to diagnostic agents that are photoactive or contrast agents. Photoactive compounds can comprise compounds such as chromagens or dyes. Contrast agents may be, for example a paramagnetic ion, wherein the ion comprises a metal selected from the group of chromium (III); manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III). The contrast agent may also be a radio-opaque compound used in X-ray techniques or computed tomography, such as an iodine, iridium, barium, gallium and thallium compound. Radio-opaque compounds may be selected from the group of barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride. In some embodiments, the diagnostic immunoconjugates may contain ultrasound-enhancing agents such as a gas filled liposome that is conjugated to an antibody of the invention. Diagnostic immunoconjugates may be used for a variety of procedures including, but not limited to, intraoperative, endoscopic or intravascular methods of tumor or cancer diagnosis and detection.

In some embodiments antibody conjugates are made using a variety of bifunctional protein coupling agents such as N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used. Agents may be additionally linked to the antibodies of the invention through a carbohydrate moiety.

In some embodiments fusion proteins comprising the antibodies of the invention and cytotoxic agents may be made, e.g. by recombinant techniques or peptide synthesis. In some embodiments such immunoconjugates comprising the anti-tumor antigen antibody conjugated with a cytotoxic agent are administered to the patient. In some embodiments the immunoconjugate and/or tumor cell antigen protein to which it is bound is/are internalized by the cell, resulting in increased therapeutic efficacy of the immunoconjugate in killing the cancer cell to which it binds. In some embodiments, the cytotoxic agent targets or interferes with nucleic acid in the cancer cell. Examples of such cytotoxic agents include maytansinoids, calicheamicins, ribonucleases and DNA endonucleases.

In some embodiments the antibodies are conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

In some embodiments the antibodies are conjugated to a cytotoxic molecule which is released inside a target cell lysozome. For example, the drug monomethyl auristatin E (MMAE) can be conjugated via a valine-citrulline linkage which will be cleaved by the proteolytic lysozomal enzyme cathepsin B following internalization of the antibody conjugate (see for example WO03/026577 published Apr. 3, 2003). In some embodiments, the MMAE can be attached to the antibody using an acid-labile linker containing a hydrazone functionality as the cleavable moiety (see for example WO02/088172 published Nov. 11, 2002).

Antibody Dependent Enzyme Mediated Prodrug Therapy (ADEPT)

In some embodiments the antibodies of the present invention may be used in ADEPT by conjugating the antibody to a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

In some embodiments the enzyme component of the immunoconjugate useful for ADEPT includes any enzyme capable of acting on a prodrug in such a way so as to convert it into its more active, cytotoxic form.

Enzymes that are useful in ADEPT include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; .beta.-lactamase useful for converting drugs derivatized with .beta.-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. In some embodiments antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

In some embodiments the ADEPT enzymes can be covalently bound to the antibodies by techniques well known in the art such as the use of the heterobifunctional crosslinking reagents discussed above. In some embodiments, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984).

In some embodiments identification of an antibody that acts in a cytostatic manner rather than a cytotoxic manner can be accomplished by measuring viability of a treated target cell culture in comparison with a non-treated control culture. Viability can be detected using methods known in the art such as the CellTiter-Blue® Cell Viability Assay or the CellTiter-Glo® Luminescent Cell Viability Assay (Piomega, catalog numbers G8080 and G5750 respectively). In some embodiments an antibody is considered as potentially cytostatic if treatment causes a decrease in cell number in comparison to the control culture without any evidence of cell death as measured by the means described above.

In some embodiments an in vitro screening assay can be performed to identify an antibody that promotes ADCC using assays known in the art. One exemplary assay is the In Vitro ADCC Assay. To prepare chromium 51-labeled target cells, tumor cell lines are grown in tissue culture plates and harvested using sterile 10 mM EDTA in PBS. The detached cells are washed twice with cell culture medium. Cells (5×10⁶) are labeled with 200 μCi of chromium 51 (New England Nuclear/DuPont) at 37° C. for one hour with occasional mixing. Labeled cells were washed three times with cell culture medium, then are resuspended to a concentration of 1×10⁵ cells/mL. Cells are used either without opsonization, or are opsonized prior to the assay by incubation with test antibody at 100 ng/mL and 1.25 ng/mL in PBMC assay or 20 ng/mL and 1 ng/mL in NK assay. Peripheral blood mononuclear cells are prepared by collecting blood on heparin from normal healthy donors and diluted with an equal volume of phosphate buffered saline (PBS). The blood is then layered over LYMPHOCYTE SEPARATION MEDIUM® (LSM: Organon Teknika) and centrifuged according to the manufacturer's instructions. Mononuclear cells are collected from the LSM-plasma interface and are washed three times with PBS. Effector cells are suspended in cell culture medium to a final concentration of 1×10⁷ cells/mL. After purification through LSM, natural killer (NK) cells are isolated from PBMCs by negative selection using an NK cell isolation kit and a magnetic column (Miltenyi Biotech) according to the manufacturer's instructions. Isolated NK cells are collected, washed and resuspended in cell culture medium to a concentration of 2×10⁶ cells/mL. The identity of the NK cells is confirmed by flow cytometric analysis. Varying effector:target ratios are prepared by serially diluting the effector (either PBMC or NK) cells two-fold along the rows of a microtiter plate (100 μL final volume) in cell culture medium. The concentration of effector cells ranges from 1.0×10⁷/mL to 2.0×10⁴/mL for PBMC and from 2.0×10⁶/mL to 3.9×10³/mL for NK. After titration of effector cells, 100 μL of chromium 51-labeled target cells (opsonized or nonoponsonized) at 1×10⁵ cells/mL are added to each well of the plate. This results in an initial effector:target ratio of 100:1 for PBMC and 20:1 for NK cells. All assays are run in duplicate, and each plate contains controls for both spontaneous lysis (no effector cells) and total lysis (target cells plus 100 μL 1% sodium dodecyl sulfate, 1 N sodium hydroxide). The plates are incubated at 37° C. for 18 hours, after which the cell culture supernatants are harvested using a supernatant collection system (Skatron Instrument, Inc.) and counted in a Minaxi auto-gamma 5000 series gamma counter (Packard) for one minute. Results are then expressed as percent cytotoxicity using the formula: % Cytotoxicity=(sample cpm-spontaneous lysis)/(total lysis-spontaneous lysis)×100.

To identify an antibody that promotes CDC, the skilled artisan may perform an assay known in the art. One exemplary assay is the In Vitro CDC assay. In vitro CDC activity can be measured by incubating tumor cell antigen expressing cells with human (or alternate source) complement-containing serum in the absence or presence of different concentrations of test antibody. Cytotoxicity is then measured by quantifying live cells using ALAMAR BLUE® (Gazzano-Santoro et al., J. Immunol. Methods 202 163-171 (1997)). Control assays are performed without antibody, and with antibody, but using heat inactivated serum and/or using cells which do not express the tumor cell antigen in question. Alternatively, red blood cells can be coated with tumor antigen or peptides derived from tumor antigen, and then CDC may be assayed by observing red cell lysis (see for example Karjalainen and Mantyjarvi, Acta Pathol Microbiol Scand [C]. 1981 October; 89(5):315-9).

To select for antibodies that induce cell death, loss of membrane integrity as indicated by, e.g., PI, trypan blue or 7AAD uptake may be assessed relative to control. One exemplary assay is the PI uptake assay using tumor antigen expressing cells. According to this assay, tumor cell antigen expressing cells are cultured in Dulbecco's Modified Eagle Medium (D-MEM):Ham's F-12 (50:50) supplemented with 10% heat-inactivated FBS (Hyclone) and 2 mM L-glutamine. (Thus, the assay is performed in the absence of complement and immune effector cells). The tumor cells are seeded at a density of 3×10⁶ per dish in 100×20 mm dishes and allowed to attach overnight. The medium is then removed and replaced with fresh medium alone or medium containing 10 μg/mL of the appropriate monoclonal antibody. The cells are incubated for a 3 day time period. Following each treatment, monolayers are washed with PBS and detached by trypsinization. Cells are then centrifuged at 1200 rpm for 5 minutes at 4° C., the pellet resuspended in 3 mL ice cold Ca²⁺ binding buffer (10 mM Hepes, pH 7.4, 140 mM NaCl, 2.5 mM CaCl₂) and aliquoted into 35 mm strainer-capped 12×75 tubes (1 mL per tube, 3 tubes per treatment group) for removal of cell clumps. Tubes then receive PI (10 μg/mL). Samples may be analyzed using a FACSCAN™ flow cytometer and FACSCONVERT™. CellQuest software (Becton Dickinson). Those antibodies that induce statistically significant levels of cell death as determined by PI uptake may be selected as cell death-inducing antibodies.

Antibodies can also be screened in vivo for apoptotic activity using ¹⁸F-annexin as a PET imaging agent. In this procedure, Annexin V is radiolabeled with ¹⁸F and given to the test animal following dosage with the antibody under investigation. One of the earliest events to occur in the apoptotic process is the eversion of phosphatidylserine from the inner side of the cell membrane to the outer cell surface, where it is accessible to annexin. The animals are then subjected to PET imaging (see Yagle et al, J Nucl Med. 2005 April; 46(4):658-66). Animals can also be sacrificed and individual organs or tumors removed and analyzed for apoptotic markers following standard protocols.

While in some embodiments cancer may be characterized by overexpression of a gene expression product, such as FGF21, the present application further provides methods for treating cancer which is not considered to be a tumor antigen-overexpressing cancer. To determine tumor antigen expression in the cancer, various diagnostic/prognostic assays are available. In some embodiments, gene expression product overexpression can be analyzed by IHC. Paraffin embedded tissue sections from a tumor biopsy may be subjected to the IHC assay and accorded a tumor antigen protein staining intensity criteria as follows:

Score 0: no staining is observed or membrane staining is observed in less than 10% of tumor cells.

Score 1+: a faint/barely perceptible membrane staining is detected in more than 10% of the tumor cells. The cells are only stained in part of their membrane.

Score 2+: a weak to moderate complete membrane staining is observed in more than 10% of the tumor cells.

Score 3+: a moderate to strong complete membrane staining is observed in more than 10% of the tumor cells.

Those tumors with 0 or 1+ scores for tumor antigen overexpression assessment may be characterized as not overexpressing the tumor antigen, whereas those tumors with 2+ or 3+ scores may be characterized as overexpressing the tumor antigen.

Alternatively, or additionally, FISH assays such as the INFORMT™ (sold by Ventana, Ariz.) or PATHVISIONT™ (Vysis, Ill.) may be carried out on formalin-fixed, paraffin-embedded tumor tissue to determine the extent (if any) of tumor antigen overexpression in the tumor.

Additionally, antibodies can be chemically modified by covalent conjugation to a polymer to increase their circulating half-life, for example. Each antibody molecule may be attached to one or more (i.e., 1, 2, 3, 4, 5 or more) polymer molecules. Polymer molecules are preferably attached to antibodies by linker molecules. The polymer may, in general, be a synthetic or naturally occurring polymer, for example an optionally substituted straight or branched chain polyalkene, polyalkenylene or polyoxyalkylene polymer or a branched or unbranched polysaccharide, e.g. homo- or hetero-polysaccharide. In some embodiments the polymers are polyoxyethylene polyols and polyethylene glycol (PEG). PEG is soluble in water at room temperature and has the general formula: R(O—CH₂—CH₂)ₙO—R where R can be hydrogen, or a protective group such as an alkyl or alkanol group. In some embodiments, the protective group has between 1 and 8 carbons. In some embodiments the protective group is methyl. The symbol n is a positive integer, between 1 and 1,000, or 2 and 500. In some embodiments the PEG has an average molecular weight between 1000 and 40,000, between 2000 and 20,000, or between 3,000 and 12,000. In some embodiments, PEG has at least one hydroxy group. In some embodiments the hydroxy is a terminal hydroxy group. In some embodiments it is this hydroxy group which is activated to react with a free amino group on the inhibitor. However, it will be understood that the type and amount of the reactive groups may be varied to achieve a covalently conjugated PEG/antibody of the present invention. Polymers, and methods to attach them to peptides, are shown in U.S. Pat. Nos. 4,766,106; 4,179,337; 4,495,285; and 4,609,546 each of which is hereby incorporated by reference in its entirety.

Safety Studies

The antibodies of the invention can be examined for safety and toxicological characteristics. Guidelines for these types of studies can be found in the document issued by the USDA CBER division, “Points to Consider in the Manufacture and Testing of Monoclonal Antibody Products for Human Use” (Docket No. 94D-0259, Feb. 28, 1997) incorporated herein by reference. In general, the candidate antibodies should be screened in preclinical studies using a number of human tissue samples and/or isolated human cell types to assess non-target tissue binding and cross reactivity. Following a satisfactory outcome from these human tissue studies, a panel of tissue samples or isolated cells from a variety of animal species can be screened to identify a suitable species for use in general toxicological studies. If no cross reactive animal species is identified, other types of models may be deemed appropriate. These other models can include studies such as xenograft models, where human tumor cells are implanted into a rodent host, or the use of a surrogate monoclonal antibody which recognizes the corresponding tumor-cell antigen in the animal species chosen for the toxicological studies. It should be appreciated that the data from these types of alternate models will be first approximations and proceeding into higher species should be done with caution.

For a candidate naked antibody, studies looking at simple tolerability can be performed. In these studies the therapeutic index of the candidate molecule can be characterized by observing any dose-dependent pharmacodynamic effects. A broad range of doses should be use (for example from 0.1 mg/kg to 100 mg/kg). Differences between tumor cell antigen number, affinity of the candidate antibody for the cross reactive animal target and differences in cellular response following binding of the antibody should be considered in estimating therapeutic index. Pharmacodynamic and pharmacokinetic studies should also be carried out in an appropriate animal model to help guild initial dose considerations when the candidate antibody is tested in humans.

For candidate immunoconjugates, stability studies of the conjugate must be performed in vivo. Optimally, pharmacodynamic and pharmacokinetic studies should be carried out on the individual components of the immunoconjugate to determine the consequences of any breakdown products from the candidate immunoconjugate. Pharmacodynamic and pharmacokinetic studies should also be carried out as above in an appropriate animal model to help guild initial dose considerations. Additional consideration must be given to safety study design when the drug will be given in combination with pretreatment with naked antibody. Safety studies must be carried out with the naked antibody alone, and studies must be designed with the immunoconjugate keeping in mind that the ultimate doses of immunoconjugate will be lower in this type of treatment regimen.

For radio-immunoconjugates, animal tissue distribution studies should be carried out to determine biodistribution data. In addition, an accounting of metabolic degradation of the total dose of administered radioactivity should be performed with both early and late time points being taken. Radio-immunoconjugates can be tested for stability in vitro using serum or plasma, and methods should be developed to measure the percentages of free radionuclide, radio-immunoconjugate and labeled, non-antibody compounds.

Oligonucleotides

In some embodiments, the FGF21 modulator is an oligonucleotide. In some embodiments the oligonucleotide is an antisense or RNAi oligonucleotide. In some embodiments the oligonucleotide is complementary to a region, domain, portion, or segment of the FGF21 gene or gene product. In some embodiments, the oligonucleotide comprises from about 5 to about 100 nucleotides, from about 10 to about 50 nucleotides, from about 12 to about 35, and from about 18 to about 25 nucleotides. In some embodiments, the oligonucleotide is at least 50%, at least 75%, at least 80%, at least 90%, at least 95%, at least 96%, at least 97%, at least 98%, at least 99%, or 100% homologous to a region, portion, domain, or segment of the FGF21 gene or gene product. In some embodiments there is substantial or complete sequence homology over at least 15, 20, 25, 30, 35, 40, 50, or 100 consecutive nucleotides of the FGF21 gene or gene product. In some embodiments there is substantial or complete sequence homology over the entire length of the FGF21 gene or. gene product. In some embodiments, the oligonucleotide binds under moderate or stringent hybridization conditions to a nucleic acid molecule having a nucleotide sequence of SEQ ID NO:1.

In some embodiments, the FGF21 modulator is a double stranded RNA (dsRNA) molecule and works via RNAi (RNA interference). In some embodiments, one strand of the dsRNA is at least 80%, at least 90%, at least 95%, at least 96%, at least 97%, at least 98%, at least 99%, or 100% homologous to a region, portion, domain, or segment of the FGF21 gene. In some embodiments there is substantial or complete sequence homology over at least 15, 20, 25, 30, 35, 40, 50, 100, 200, 300, 400, 500, or 1000 consecutive nucleotides of the FGF21 gene. In some embodiments there is substantial sequence homology over the entire length of the FGF21 gene.

In some embodiments, the FGF21 modulator hybridizes to a region of an FGF21 RNA that includes a SNP. In some embodiments, the SNP includes nucleotide substitutions at one or more positions corresponding to nucleotide position 36, 325, 326, 420, 516, 521, or 621 of SEQ ID NO:1. In some embodiments, the FGF21 modulator hybridizes to a region of an FGF21 RNA including a SNP listed in Table 1.

In some embodiments oligonucleotides of the invention are used in polymerase chain reaction (PCR). This sequence may be based on (or designed from) a genomic sequence or cDNA sequence and is used to amplify, confirm, or detect the presence of an identical, similar, or complementary DNA or RNA in a particular cell or tissue.

Small Molecules

In some embodiments, the FGF21 modulator is a small molecule. As used herein, the term “small molecule” refers to an organic or inorganic non-polymer compound that has a molecular weight that is less than about 10 kilodaltons. Examples of small molecules include peptides, oligonucleotides, organic compounds, inorganic compounds, and the like. In some embodiments, the small molecule has a molecular weight that is less than about 9, about 8, about 7, about 6, about 5, about 4, about 3, about 2, or about 1 kilodalton.

Mimetics

In some embodiments, the FGF21 modulator is a mimetic. As used herein, the term “mimetic” is used to refer to compounds which mimic the activity of a peptide. Mimetics are non-peptides but may comprise amino acids linked by non-peptide bonds. U.S. Pat. No. 5,637,677, issued on Jun. 10, 1997, and parent applications thereof, all of which are incorporated herein by reference, contain detailed guidance on the production of mimetics. Briefly, the three-dimensional structure of the peptides which specifically interacts with the three dimensional structure of the FGF21 is duplicated by a molecule that is not a peptide. In some embodiments the FGF21 mimetic is a mimetic of FGF21 or a mimetic of a ligand of FGF21.

Decoys

In some embodiments, the FGF21 modulator is a decoy comprising at least a portion of an FGF21 polypeptide. In some embodiments the decoy competes with natural FGF21 polypeptides for binding to an FGF21 receptor such as FGFR-1 or FGFR-2. In some embodiments, the decoy is labeled to facilitate quantification, qualification, and/or visualization. In other embodiments, the decoy further comprises a moiety to facilitate isolation and/or separation of the decoy or the decoy-FGF21 receptor complex, such as a decoy-FGFR-1 or decoy-FGFR-2 complex. In some embodiments the decoy comprises at least a portion of an FGF21 polypeptide fused to an antibody or antibody fragment.

Methods of Treating/Preventing Cancer

The present invention provides methods for treating and/or preventing cancer or symptoms of cancer in a subject comprising administering to the subject a therapeutically effective amount of one or more FGF21 modulators of the present invention. In some embodiments the cancer is a cancer associated with overexpression of FGF21. In some embodiments, the cancer is colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma or melanoma. In some embodiments, the cancer is in a non-hormonally regulated tissue. In some embodiments the subject has been diagnosed as having a cancer or as being predisposed to cancer.

Symptoms of cancer are well-known to those of skill in the art and include, without limitation, weight loss, anemia, abdominal pain, intestinal obstruction, blood in the stool, diarrhea, constipation, other changes in bowel habits, colon metastases, death, weakness, excessive fatigue, difficulty eating, loss of appetite, chronic cough, worsening breathlessness, coughing up blood, blood in the urine, nausea, vomiting, liver metastases, lung metastases, bone metastases, abdominal fullness, bloating, fluid in peritoneal cavity, vaginal bleeding, abdominal distension, perforation of colon, acute peritonitis (infection, fever, pain), pain, vomiting blood, heavy sweating, fever, high blood pressure, jaundice, dizziness, chills, muscle spasms, lung metastases, bladder metastases, liver metastases, bone metastases, kidney metastases, and pancreas metastases, difficulty swallowing, and the like.

A therapeutically effective amount of the modulating compound can be determined empirically, according to procedures well known to medicinal chemists, and will depend, inter alia, on the age of the patient, severity of the condition, and on the ultimate pharmaceutical formulation desired. Administration of the modulators of the present invention can be carried out, for example, by inhalation or suppository or to mucosal tissue such as by lavage to vaginal, rectal, urethral, buccal and sublingual tissue, orally, topically, intranasally, intraperitoneally, parenterally, intravenously, intralymphatically, intratumorly, intramuscularly, interstitially, intra-arterially, subcutaneously, intraoccularly, intrasynovial, transepithelial, and transdermally. In some embodiments, the inhibitors are administered by lavage, orally or inter-arterially. Other suitable methods of introduction can also include rechargeable or biodegradable devices and slow or sustained release polymeric devices. As discussed above, the therapeutic compositions of this invention can also be administered as part of a combinatorial therapy with other known anti-cancer agents or other known anti-bone disease treatment regimen.

The present invention further provides methods of modulating an FGF21-related biological activity in a patient. The methods comprise administering to the patient an amount of an FGF21 modulator effective to modulate one or more FGF21 biological activities. Suitable assays for measuring FGF21 biological activities are set forth supra and infra.

The present invention also provides methods of inhibiting cancer cell growth in a patient in need thereof comprising administering a therapeutically effective amount of one or more FGF21 modulators to the patient. Suitable assays for measuring FGF21-related cell growth are known to those skilled in the art and are set forth supra and infra.

The present invention also provides methods of inhibiting cancer in a patient in need thereof. The methods comprise determining if the patient is a candidate for FGF21 therapy as described herein and administering a therapeutically effective amount of one or more FGF21 modulators to the patient if the patient is a candidate for FGF21 therapy. If the patient is not a candidate for FGF21 therapy, the patient is treated with conventional cancer treatment.

The present invention also provides methods of inhibiting cancer in a patient diagnosed or suspected of having a cancer. The methods comprise administering a therapeutically effective amount of one or more FGF21 modulators to the patient.

The present invention also provides methods for inhibiting the interaction of two or more cells in a patient comprising administering a therapeutically effective amount of an FGF21 modulator to said patient. Suitable assays for measuring FGF21-related cell interaction are known to those skilled in the art and are set forth supra and infra.

The present invention also provides methods of modulating one or more symptoms of cancer in a patient comprising administering to said patient a therapeutically effective amount of the FGF21 compositions described herein.

The present invention further provides methods for inhibiting cell growth in a patient in need thereof comprising administering to the patient a therapeutically effective amount of an FGF21 modulator. Suitable assays for measuring FGF21-related anchorage-independent cell growth are set forth supra and infra.

The present invention also provides methods for inhibiting migration of cancer cells in a patient in need thereof comprising administering to the patient a therapeutically effective amount of an FGF21 modulator. Suitable assays for measuring FGF21-related cell migration are known to those skilled in the art.

The present invention also provides methods for inhibiting adhesion of cancer cells in a patient in need thereof comprising administering to the patient a therapeutically effective amount of an FGF21 modulator. Suitable assays for measuring FGF21-related cell adhesion are known to those skilled in the art.

The present invention also provides methods of inhibiting angiogenesis in a patient in need thereof comprising administering a therapeutically effective amount of one or more FGF21 modulators to the patient. Suitable assays for measuring angiogenesis are known to those skilled in the art and are set forth infra.

The present invention also provides methods to prophylactically treat a patient who is predisposed to develop cancer, a cancer metastasis or who has had a metastasis and is therefore susceptible to a relapse or recurrence. The methods are particularly useful in high-risk individuals who, for example, have a family history of cancer or of metastasizing tumors, or show a genetic predisposition for a cancer metastasis. In some embodiments the tumors are FGF21-related tumors. Additionally, the methods are useful to prevent patients from having recurrences of FGF21-related tumors who have had FGF21-related tumors removed by surgical resection or treated with a conventional cancer treatment.

The present invention also provides methods of inhibiting cancer progression and/or causing cancer regression comprising administering to the patient a therapeutically effective amount of an FGF21 modulator.

In some embodiments, the patient in need of anti-cancer treatment is treated with the FGF21 modulators of the present invention in conjunction with chemotherapy and/or radiation therapy. For example, following administration of the FGF21 modulators, the patient may also be treated with a therapeutically effective amount of anti-cancer radiation. In some embodiments chemotherapeutic treatment is provided in combination with FGF21 modulators. In some embodiments FGF21 modulators are administered in combination with chemotherapy and radiation therapy.

Methods of treatment comprise administering single or multiple doses of one or more FGF21 modulators to the patient. In some embodiments the FGF21 modulators are administered as injectable pharmaceutical compositions that are sterile, pyrogen free and comprise the FGF21 modulators in combination with a pharmaceutically acceptable carrier or diluent.

In some embodiments, the therapeutic regimens of the present invention are used with conventional treatment regimens for cancer including, without limitation, surgery, radiation therapy, hormone ablation and/or chemotherapy. Administration of the FGF21 modulators of the present invention may take place prior to, simultaneously with, or after conventional cancer treatment. In some embodiments, two or more different FGF21 modulators are administered to the patient.

In some embodiments the amount of FGF21 modulator administered to the patient is effective to inhibit one or more of cancer cell growth, tumor formation, cancer cell proliferation, cancer cell metastasis, cell migration, angiogenesis, FGF21 signaling, FGF21-mediated cell-cell adhesion, blood vessel formation, kinase activity, cancer cell survival, glucose uptake into adipocytes, interactions between FGF21 and an FGF21 receptor, phosphorylation of an FGF21 receptor, and FGF21 expression. The FGF21 receptor can be FGFR-1 or FGFR-2. In some embodiments the amount of FGF21 modulator administered to the patient is effective to increase cancer cell death through apoptosis.

Combination Therapy

In some embodiments the invention provides compositions comprising two or more FGF21 modulators to provide still improved efficacy against cancer and/or vascular disease. In some embodiments the FGF21 modulators are monoclonal antibodies. Compositions comprising two or more FGF21 antibodies may be administered to persons or mammals suffering from, or predisposed to suffer from, cancer or vascular disease. One or more antibodies may also be administered with another therapeutic agent, such as a cytotoxic agent, or chemotherapeutic. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

In some embodiments, administration of combinations, or “cocktails”, of different antibodies is contemplated. Such antibody cocktails may have certain advantages inasmuch as they contain antibodies which exploit different effector mechanisms or combine directly cytotoxic antibodies with antibodies that rely on immune effector functionality. Such antibodies in combination may exhibit synergistic therapeutic effects.

A cytotoxic agent refers to a substance that inhibits or prevents the function of cells and/or causes destruction of cells. The term is intended to include radioactive isotopes (e.g., ¹³¹I, ¹²⁵I, ⁹⁰Y and ¹⁸⁶Re), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with an antibody according to the invention.

In some embodiments, conventional cancer medicaments are administered with the compositions of the present invention. Conventional cancer medicaments include:

a) cancer chemotherapeutic agents;

b) additional agents;

c) prodrugs.

Cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, caminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine).

Additional agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

“Prodrug” refers to a precursor or derivative form of a pharmaceutically active substance that is less cytotoxic or non-cytotoxic to tumor cells compared to the parent drug and is capable of being enzymatically activated or converted into an active or the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, b-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

Clinical Aspects

In some embodiments, the methods and compositions of the present invention are particularly useful in colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma and melanoma. In some embodiments, the cancer is ductal adenocarcinoma, lobular adenocarcinoma, or metastatic adenocarcinoma.

Methods of Treating/Preventing Vascular Disease

The present invention provides methods for treating and/or preventing vascular disease or symptoms of vascular disease in a subject comprising administering to the subject a therapeutically effective amount of one or more FGF21 modulators of the present invention. In some embodiments, the vascular disease is coronary artery disease, peripheral artery disease, abdominal aortic aneurysm, blood clots, deep vein thrombosis, venous stasis disease, phlebitis, or varicose veins. In some embodiments the subject has been diagnosed as having a vascular disease or as being predisposed to vascular disease. In some embodiments, the subject has atherosclerosis.

Symptoms of vascular disease are well-known to those of skill in the art and include, without limitation, intermittent claudication, angina, ischemic rest pain, ulcers, gangrene, withered muscles, pain, hair loss over toes and legs, thick toenails, shiny skin, chest pain, shortness of breath, and the like.

A therapeutically effective amount of the modulating compound can be determined empirically, according to procedures well known to medicinal chemists, and will depend, inter alia, on the age of the patient, severity of the condition, and on the ultimate pharmaceutical formulation desired. Administration of the modulators of the present invention can be carried out, for example, by inhalation or suppository or to mucosal tissue such as by lavage to rectal, urethral, buccal and sublingual tissue, orally, topically, intranasally, intraperitoneally, parenterally, intravenously, intralymphatically, intramuscularly, interstitially, intra-arterially, subcutaneously, intraoccularly, intrasynovial, transepithelial, and transdermally. In some embodiments, the inhibitors are administered by lavage, orally or inter-arterially. Other suitable methods of introduction can also include rechargeable or biodegradable devices and slow or sustained release polymeric devices. As discussed above, the therapeutic compositions of this invention can also be administered as part of a combinatorial therapy with other known agents or another treatment regimen for treating vascular disease.

The present invention also provides methods of increasing angiogenesis in a patient in need thereof comprising administering a therapeutically effective amount of one or more FGF21 modulators to the patient. Suitable assays for measuring angiogenesis are known to those skilled in the art and include microscopy assays, and immunohistochemistry to detect von Willebrand Factor or CD31 as positive markers of angiogenesis (see, e.g., Auerbach, Clinical Chemistry 49:1, 32-40, 2003; Taraboletti and Giavazzi, EJC 40:881-889, 2004).

The present invention further provides methods of inhibiting vascular disease in a patient diagnosed or suspected of having a vascular disease. The methods comprise administering a therapeutically effective amount of one or more FGF21 modulators to the patient.

The present invention also provides methods for increasing blood vessel formation in a patient comprising administering a therapeutically effective amount of an FGF21 modulator to said patient. Suitable assays for measuring blood vessel formation are known to those skilled in the art and include assays for measuring angiogenesis described supra, as well as monitoring of endothelial cell proliferation (WO 01/63281).

The present invention also provides methods of modulating one or more symptoms of vascular disease in a patient comprising administering to said patient a therapeutically effective amount of the FGF21 compositions described herein.

The present invention also provides methods to prophylactically treat a patient who is predisposed to develop a vascular disease. The methods are particularly useful in high-risk individuals who, for example, have a family history of vascular disease, or show a genetic predisposition for a vascular disease.

The present invention also provides methods of inhibiting progression of vascular disease comprising administering to the patient a therapeutically effective amount of an FGF21 modulator.

In some embodiments, the patient in need of treatment for vascular disease is treated with the FGF21 modulators of the present invention in conjunction with surgery, medication, or a special diet or exercise regimen. For example, following administration of the FGF21 modulators, the patient may also be treated with a therapeutically effective amount of an antiplatelet agent, anticoagulant, or thrombolytic. In some embodiments, a subject is treated by angioplasty, stenting, atherectomy, or bypass surgery prior to and/or following treatment with an FGF21 modulator. Administration of the FGF21 modulators of the present invention may take place prior to, simultaneously with, or after conventional treatment of vascular disease. In some embodiments, two or more different FGF21 modulators are administered to the patient.

Pharmaceutical Compositions

The present invention also provides pharmaceutical compositions comprising one or more of the FGF21 modulators described herein and a pharmaceutically acceptable carrier. In some embodiments the pharmaceutical compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

Methods of Detecting FGF21

The present invention also provides methods for detecting FGF21. In some embodiments the FGF21 is present in a patient or in a patient sample. In some embodiments the method comprises administering a composition comprising one or more FGF21 modulators to the patient and detecting the localization of the imaging agent in the patient. In some embodiments the patient sample comprises cancer cells. In some embodiments the FGF21 modulator is linked to an imaging agent or is detectably labeled. In some embodiments, the FGF21 modulator is an FGF21 antibody conjugated to an imaging agent and is administered to a patient to detect one or more tumors or to determine susceptibility of the patient to FGF21 therapy. The labeled antibodies will bind to the high density of receptors on cells and thereby accumulate on the tumor cells. Using standard imaging techniques, the site of the tumors can be detected.

The present invention also provides methods of imaging/detecting cells or tumors expressing or overexpressing FGF21 comprising contacting a composition comprising an FGF21 modulator to a sample and detecting the presence of the FGF21 modulator in the sample. In some embodiments the sample is a patient sample. In some embodiments the patient sample comprises cancer cells. In some embodiments the FGF21 modulator is linked to an imaging agent or is detectably labeled.

The present invention also provides methods for quantifying the amount of FGF21 present in a patient, cell or sample. The methods comprise administering one or more of antibodies, probes, or small molecules to a patient or sample and detecting the amount of FGF21 present in the sample. In some embodiments the antibodies, probes, or small molecules are linked to an imaging agent or are detectably labeled. Such information indicates, for example, whether or not a tumor is related to FGF21, and, therefore, whether specific treatments should be used or avoided. In some embodiments, using standard techniques well known to the art-skilled, samples believed to include tumor cells are obtained and contacted with labeled antibodies, probes, oligonucleotides, and small molecules. After removing any unbound, labeled antibodies, probes, oligonucleotides or small molecules, the quantity of labeled antibodies, peptides, oligonucleotides or mimetics bound to the cell, or the quantity of antibodies, peptides, oligonucleotides or mimetics removed as unbound is determined. The information directly relates to the amount of FGF21 present.

Imaging can be performed using procedures well known to those of ordinary skill in the art. Imaging can be performed, for example, by radioscintigraphy, nuclear magnetic resonance imaging (MRI) or computed tomography (CT scan). The most commonly employed radiolabels for imaging agents include radioactive iodine and indium. Imaging by CT scan may employ a heavy metal such as an iron chelate. MRI scanning may employ chelates of gadolinium or manganese. Additionally, positron emission tomography (PET) may be possible using positron emitters of oxygen, nitrogen, iron, carbon, or gallium.

In some embodiments the FGF21 modulator is an FGF21 antibody. In some embodiments the modulator is linked to an imaging agent or is detectably labeled. In some embodiments the imaging agent is ¹⁸F, ⁴³K, ⁵²Fe, ⁵⁷Co, ⁶⁷Cu, ⁶⁷Ga, ⁷⁷Br, ⁸⁷MSr, ⁸⁶Y, ⁹⁰Y, ⁹⁹MTc, ¹¹¹In, ¹²³I, ¹²⁵I, ¹²⁷Cs, ¹²⁹Cs, ¹³¹I, ¹³²I, ¹⁹⁷Hg, ²⁰³Pb, or ²⁰⁶Bi.

Methods of detection are well known to those of skill in the art. For example, methods of detecting polynucleotides include, but are not limited to PCR, Northern blotting, Southern blotting, RNA protection, and DNA hybridization (including in situ hybridization). Methods of detecting polypeptides include, but are not limited to, Western blotting, ELISA, enzyme activity assays, slot blotting, peptide mass fingerprinting, electrophoresis, immunochemistry and immunohistochemistry. Other examples of detection methods include, but are not limited to, radioimmunoassay (RIA), chemiluminescence immunoassay, fluoroimmunoassay, time-resolved fluoroimmunoassay (TR-FIA), two color fluorescent microscopy, or immunochromatographic assay (ICA), all well known by those of skill in the art. In some preferred embodiments of the present invention, polynucleotide expression is detected using PCR methodologies and polypeptide production is detected using ELISA technology.

Methods for Delivering a Cytotoxic Agent or a Diagnostic Agent to a Cell

The present invention also provides methods for delivering a cytotoxic agent or a diagnostic agent to one or more cells that express FGF21. In some embodiments the methods comprise contacting an FGF21 modulator of the present invention conjugated to a cytotoxic agent or diagnostic agent with the cell.

Methods for Determining Susceptibility to FGF21 Therapy

The present invention also provides methods for determining the susceptibility of a patient to FGF21 therapy. The methods comprise detecting the presence or absence of evidence of differential expression of FGF21 in a patient or patient sample. The presence of evidence of differential expression of FGF21 in the patient or sample is indicative of a patient who is susceptible to FGF21 therapy. In some embodiments, the absence of evidence of differential expression of FGF21 in the patient or patient sample is indicative of a patient who is not a candidate for FGF21 therapy.

In some embodiments the therapeutic methods comprise first identifying patients susceptible to FGF21 therapy comprising administering to the patient in need thereof a composition comprising an FGF21 modulator linked to an imaging agent and detecting the presence or absence of evidence of an FGF21 RNA or protein in the patient. In some embodiments, the therapeutic methods further comprise administering one or more FGF21 modulators to the patient if the patient is a candidate for FGF21 therapy and treating the patient with conventional treatment if the patient is not a candidate FGF21 therapy.

In some embodiments, the therapeutic methods comprise first identifying patients susceptible to FGF21 therapy comprising assaying for the presence or absence of a SNP in the FGF21 gene of the patient. The SNP can be one or more SNPs listed in Table 1. The presence or absence of a SNP can be determined by any known method, including restriction fragment length polymorphism (RFLP), restriction site generating PCR, DNA sequencing, and the like. In some embodiments, the presence of a SNP can indicate that a patient has an FGF21 related cancer or disorder and/or is a candidate for treatment with an FGF21 modulator.

In some embodiments, a patient is identified as a candidate for treatment with an FGF21 modulator if the patient is determined to carry an FGF21 polypeptide that has an amino acid other than leucine at a position corresponding to position 174 of SEQ ID NO:2. In some embodiments, the patient has a proline at position 174.

In some therapeutic methods, one or more FGF21 modulators are administered to the patients alone or in combination with other anti-cancer medicaments when the patient is identified as having a cancer or being susceptible to a cancer.

In some therapeutic methods, one or more FGF21 modulators are administered to the patients alone or in combination with other medicaments for treating vascular disease when the patient is identified as having a vascular disease or being susceptible to a vascular disease.

Methods for Assessing the Progression of Cancer

The invention also provides methods for assessing the progression of cancer in a patient comprising comparing the level of an expression product of FGF21 in a biological sample at a first time point to a level of the same expression product at a second time point. A change in the level of the expression product at the second time point relative to the first time point is indicative of the progression of the cancer.

Methods of Screening

The present invention also provides methods of screening for anti-cancer agents or angiogenic or anti-angiogenic agents. The methods comprise contacting a cell expressing FGF21 with a candidate compound and determining whether an FGF21-related biological activity is modulated. In some embodiments, inhibition of one or more of cancer cell growth, integrin mediated activities, tumor formation, cancer cell proliferation, cancer cell metastasis, cell migration, angiogenesis, FGF21 signaling, FGF21-mediated cell-cell adhesion, uptake of glucose into adipocytes, interactions between FGF21 and one or both of FGFR-1 or FGFR-2, phosphorylation of FGFR-1 or FGFR-2 and FGF21 expression is indicative of an anti-cancer agent. In other embodiments, an increase of one or more of endothelial cell proliferation, angiogenesis, blood vessel formation, FGF21 signaling, FGF21-mediated cell-cell adhesion, uptake of glucose into adipocytes, interactions between FGF21 and one or both of FGFR-1 or FGFR-2, phosphorylation of FGFR-1 or FGFR-2, or FGF21 expression is indicative of an agent for treatment of vascular disease.

The present invention further provides methods of identifying a cancer inhibitor. The methods comprise contacting a cell expressing FGF21 with a candidate compound and an FGF21 ligand, and determining whether an FGF21-related biological activity is modulated. In some embodiments, inhibition of one or more of cancer cell growth, integrin mediated activities, tumor formation, cancer cell proliferation, cancer cell metastasis, cell migration, angiogenesis, FGF21 signaling, FGF21-mediated cell-cell adhesion, uptake of glucose into adipocytes, interactions between FGF21 and one or both of FGFR-1 or FGFR-2, phosphorylation of FGFR-1 or FGFR-2, and FGF21 expression is indicative of a cancer inhibitor. In some embodiments the amount of FGF21 modulator administered to the patient is effective to increase cancer cell apoptosis.

In some embodiments, the invention provides methods of screening for anti-cancer agents, particularly anti-metastatic cancer agents, by, for example, screening putative modulators for an ability to modulate the activity or level of a downstream marker. In some embodiments candidate agents that decrease FGFR-1 or FGFR-2 levels are identified as anti-cancer agents.

In some embodiments, the invention provides methods of screening for agents that treat vascular disease by, for example, screening putative modulators for an ability to modulate the activity or level of a downstream marker. In some embodiments candidate agents that increase FGFR-1 or FGFR-2 levels are identified as agents for treating vascular disease.

Methods for Purifying FGF21

In some embodiments, the invention provides methods of purifying FGF21 protein from a sample comprising FGF21. The methods comprise providing an affinity matrix comprising an FGF21 antibody of the present invention bound to a solid support, contacting the sample with the affinity matrix to form an affinity matrix-FGF21 protein complex, separating the affinity matrix-FGF21 protein complex from the remainder of the sample; and releasing FGF21 protein from the affinity matrix.

Kits

In some embodiments, the present invention provides kits for imaging and/or detecting a gene or gene product correlated with FGF21 overexpression. Kits of the invention comprise detectable antibodies, small molecules, oligonucleotides, decoys, mimetics or probes as well as instructions for performing the methods of the invention. Optionally, kits may also contain one or more of the following: controls (positive and/or negative), containers for controls, photographs or depictions of representative examples of positive and/or negative results.

Each of the patents, patent applications, accession numbers and publications described herein is hereby incorporated by reference in its entirety.

Various modifications of the invention, in addition to those described herein, will be apparent to those of skill in the art in view of the foregoing description. Such modifications are also intended to fall within the scope of the appended embodiments. The present invention is further demonstrated in the following examples that are for purposes of illustration and are not intended to limit the scope of the present invention.

### EXAMPLES

### Example 1

### FGF21 Stimulates Endothelial Cell Proliferation

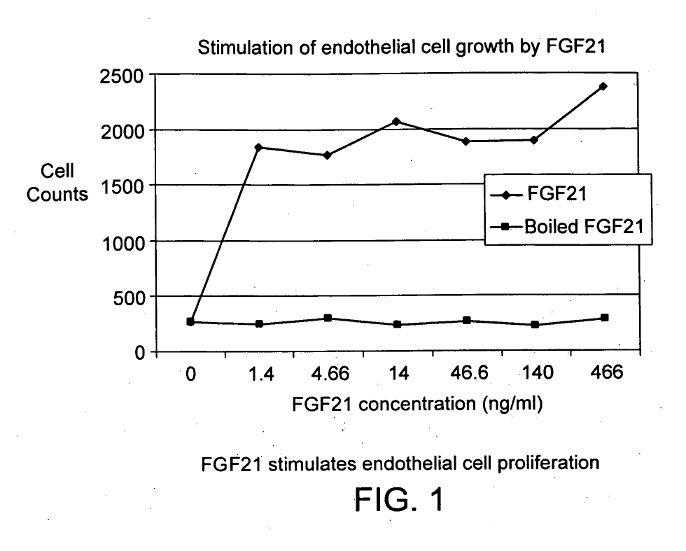

Proliferation assays were performed using Bovine Adrenal Cortex Endothelial (ACE) cells. Cells were prepared and cultured as described (Gospodarowicz et al., Proc. Natl. Acad. Sci. USA 86:7311-7315, 1989; Gospodarowicz et al., J. Cellular Physiology 127:121-136, 1986). At the time of assay, ACE cells were plated in 24-well tissue culture plates at 5,000 cells per well in serum-free media. FGF21 or boiled FGF21 were added at various concentrations ranging from 1.4 to 466 ng/mL, in duplicate, and incubations were performed for 72 hours. Following incubation, cells were harvested and counted in a Coulter cell counter. Cell counts were averaged between the duplicate samples. FGF21 stimulated cell proliferation, while boiled FGF21 had no effect on cell proliferation (FIG. 1).

### Example 2

### Colon Cancer Cell Lines Exhibit High FGF21 Expression Levels

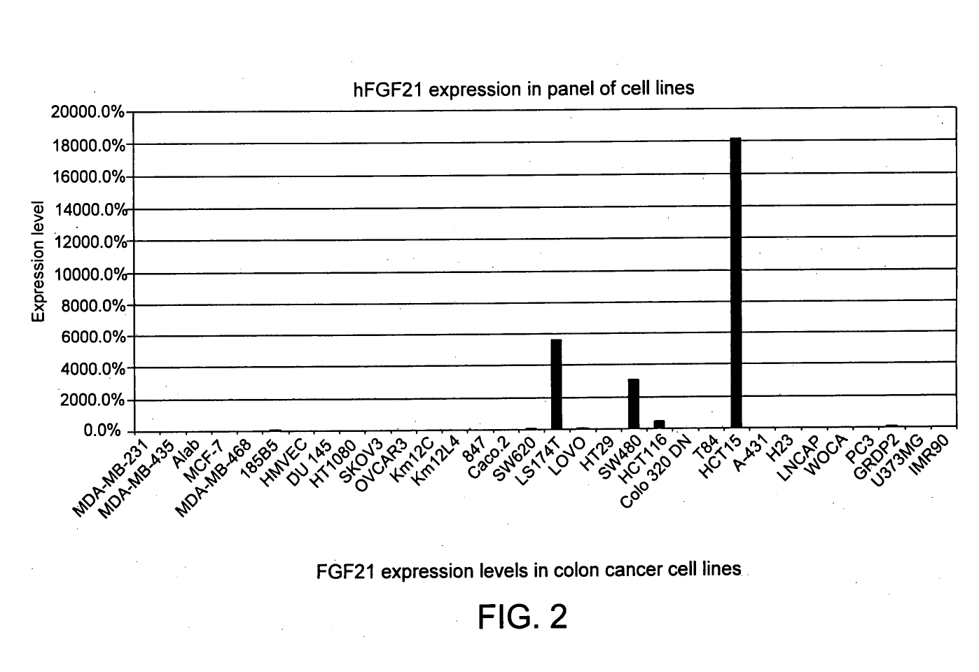

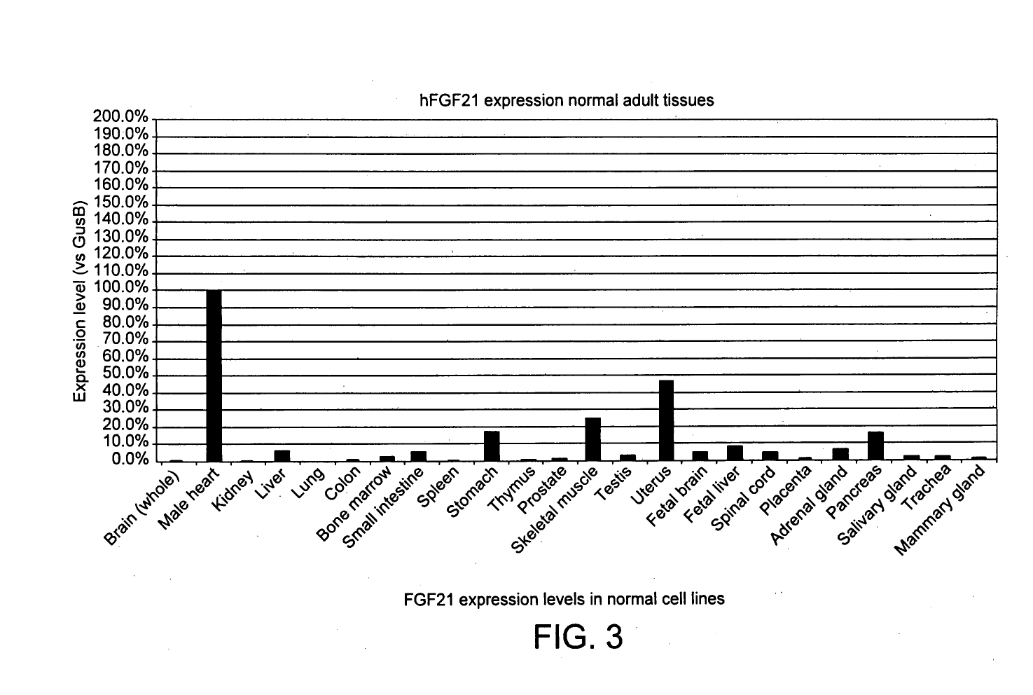

FGF21 mRNA levels were examined in normal adult tissue and in a panel of cell lines derived from cancer and normal tissues (FIGS. 2 and 3). Expression levels were assayed using real-time RT-PCR, and RNA levels were normalized against RNA levels of the housekeeping gene GusB.

Total RNA from normal human adult organs (Stratagene, La Jolla, Calif.) and total RNA from cultured cells were reverse-transcribed with oligo-dT18 primer at 42° C. for 1 hour then heated at 94° C. for 5 minutes in a total reaction volume of 20 μl (First-Strand™ cDNA Synthesis Kit, Clontech). The resulting mix was then used as template for PCR in a Lightcycler® Instrument (Roche Diagnostics Corporation, Indianapolis, Ind.).

PCR was performed using the following gene-specific primers:

Gus-B:
forward primer

| | |
|---|---|
| 5′-CCTTTTGCGAGAGAGATACT-3′ | (SEQ ID NO: 209) |
| reverse primer | |
| 5′-CCTTTAGTGTTCCCTGCTAG-3′ | (SEQ ID NO: 210) |
| FGF21: | |
| forward primer | |
| 5′-GTCCTCTCCTGCAATTCGGG-3′ | (SEQ ID NO: 211) |
| reverse primer | |
| 5′-CGTCCCATCCTCCCTGATCT-3′ | (SEQ ID NO: 212) |

The 20-μl PCR reaction mix in each Lightcycler capillary contained 2 μl of 10×PCR buffer II, 3 mM MgCl₂ (Perkin-Elmer, Foster City, Calif.), 140 μM dNTP, 1:50000 of SYBR Green I, 0.25 mg/ml BSA, 1 unit of Taq polymerase (Boehringer Mannheim, Indianapolis, Ind.), 0.175 μM each primer, 2 μl of RT reaction mix. The PCR amplification began with 20-second denaturation at 95° C., followed by 45 cycles of denaturation at 95° C. for 5 seconds, annealing at 60° C. for 1 second and extension at 72° C. for 30 seconds. At the end of the final cycle, PCR products were annealed at 60° C. for 5 seconds, then slowly heated to 95° C. at 0.2° C./second, to measure melting curves of specific PCR products. All experiments were performed in duplicate. Data analysis was performed using Lightcycler Software (Roche Diagnostics Corporation, Indianapolis, Ind.) with quantification and melting curve options.

Heart tissue was observed to express the highest level of FGF21 among normal tissue types. Expression levels were therefore assessed as a percentage of normal heart FGF21 expression. The highest FGF21 expression levels were observed in LS174T cells (human colorectal adenocarcinoma; 56-fold FGF21 expression compared to heart), SW480 cells (human colorectal adenocarcinoma; 31-fold FGF21 expression compared to heart), HCT116 (human colorectal carcinoma; 4.6-fold FGF21 expression compared to heart), and HCT15 (human colorectal adenocarcinoma; 181-fold FGF21 expression compared to heart) (FIG. 2).

### Example 3

### FGF21 Sequence Characteristics

Several SNPs have been identified in FGF21.

TABLE 1

| | Nucleotide | | | |
|---|---|---|---|---|
| GenBank | positions¹ | Nucleotide | Amino acid | Amino acid |
| reference ID | (mRNA) | change | position² | change |
| rs17851645 | 36 | A to G | 12 | None |
| rs3745712 | 325 | C to T | 109 | Ala to Thr |
| rs3745711 | 326 | G to T | 109 | Ala to Asp |
| rs3745710 | 420 | G to A | 140 | None |
| rs885662 | 516 | G to C | 172 | None |
| rs17856566 | 521 | T to C | 174 | Leu to Pro |
| rs838130 | 621 | G to A | 207 | None |

¹Nucleotide position corresponds to the position in SEQ ID NO:1
²Amino acid position corresponds to the position in SEQ ID NO:2

FGF21 (see, for example, U.S. Pat. No. 6,716,626) and FGF19x (WO 01/18209) are identical at their N-termini (amino acids 1-148) but diverge after amino acid 149 (FIG. 4). The C-terminus of FGF19x from amino acid 149 is only 5 amino acids long (LQRLL). The C-terminus of FGF21 from amino acid 149 is 60 amino acids long. The FGF21 transcript has 4 exons, and the coding region resides in exons 2-4. (Exon 2: nt 1-235 (codon 1-78); Exon 3: nt 236-339 (codon 79-112); Exon 4: nt 340-630 (codon 113-210)). FGF21 includes the entire fourth exon of the mRNA transcript, while FGF19x includes only a portion of the fourth exon. The signal peptide of both proteins is located at amino acids 1-28. The FGF21 receptor binding domain of FGF21 is at amino acids 45-165.

### Example 4

### FGF-21 Epitopes

Linear epitopes of FGF21 for antibody recognition and preparation can be identified by any of numerous methods known in the art. Some example methods include probing antibody-binding ability of peptides derived from the amino acid sequence of the antigen. Binding can be assessed by using BIACORE or ELISA methods. Other techniques include exposing peptide libraries on planar solid support (“chip”) to antibodies and detecting binding through any of multiple methods used in solid-phase screening. Additionally, phage display can be used to screen a library of peptides with selection of epitopes after several rounds of biopanning.

Table 2 below provides regions of FGF21 (SEQ ID NO:2) that have been identified as linear epitopes suitable for recognition by anti-FGF21 antibodies.

TABLE 2

| Mapped | | | | | SEQ |
|---|---|---|---|---|---|
| region | epitope | | aa seq | epitope | ID |
| (aa) | length | Epitope | location | # | NO: |
| 45-63 | 8-mer | RQRYLYTD | 45-52 | 1 | 3 |
| 45-63 | 8-mer | QRYLYTDD | 46-53 | 2 | 4 |
| 45-63 | 8-mer | RYLYTDDA | 47-54 | 3 | 5 |
| 45-63 | 8-mer | YLYTDDAQ | 48-55 | 4 | 6 |
| 45-63 | 8-mer | LYTDDAQQ | 49-56 | 5 | 7 |
| 45-63 | 8-mer | YTDDAQQT | 50-57 | 6 | 8 |
| 45-63 | 8-mer | TDDAQQTE | 51-58 | 7 | 9 |
| 45-63 | 8-mer | DDAQQTEA | 52-59 | 8 | 10 |
| 45-63 | 8-mer | DAQQTEAH | 53-60 | 9 | 11 |
| 45-63 | 8-mer | AQQTEAHL | 54-61 | 10 | 12 |
| 45-63 | 8-mer | QQTEAHLE | 55-62 | 11 | 13 |
| 45-63 | 8-mer | QTEAHLEI | 56-63 | 12 | 14 |
| 45-63 | 9-mer | RQRYLYTDD | 45-53 | 13 | 15 |
| 45-63 | 9-mer | QRYLYTDDA | 46-54 | 14 | 16 |
| 45-63 | 9-mer | RYLYTDDAQ | 47-55 | 15 | 17 |
| 45-63 | 9-mer | YLYTDDAQQ | 48-56 | 16 | 18 |
| 45-63 | 9-mer | LYTDDAQQT | 49-57 | 17 | 19 |
| 45-63 | 9-mer | YTDDAQQTE | 50-58 | 18 | 20 |
| 45-63 | 9-mer | TDDAQQTEA | 51-59 | 19 | 21 |
| 45-63 | 9-mer | DDAQQTEAH | 52-60 | 20 | 22 |
| 45-63 | 9-mer | DAQQTEAHL | 53-61 | 21 | 23 |
| 45-63 | 9-mer | AQQTEAHLE | 54-62 | 22 | 24 |
| 45-63 | 9-mer | QQTEAHLEI | 55-63 | 23 | 25 |
| 45-63 | 10-mer | RQRYLYTDDA | 45-54 | 24 | 26 |
| 45-63 | 10-mer | QRYLYTDDAQ | 46-55 | 25 | 27 |
| 45-63 | 10-mer | RYLYTDDAQQ | 47-56 | 26 | 28 |
| 45-63 | 10-mer | YLYTDDAQQT | 48-57 | 27 | 29 |
| 45-63 | 10-mer | LYTDDAQQTE | 49-58 | 28 | 30 |
| 45-63 | 10-mer | YTDDAQQTEA | 50-59 | 29 | 31 |
| 45-63 | 10-mer | TDDAQQTEAH | 51-60 | 30 | 32 |
| 45-63 | 10-mer | DDAQQTEAHL | 52-61 | 31 | 33 |
| 45-63 | 10-mer | DAQQTEAHLE | 53-62 | 32 | 34 |
| 45-63 | 10-mer | AQQTEAHLEI | 54-63 | 33 | 35 |
| 71-90 | 8-mer | GAADQSPE | 71-78 | 1 | 36 |
| 71-90 | 8-mer | AADQSPES | 72-79 | 2 | 37 |
| 71-90 | 8-mer | ADQSPESL | 73-80 | 3 | 38 |
| 71-90 | 8-mer | DQSPESLL | 74-81 | 4 | 39 |
| 71-90 | 8-mer | QSPESLLQ | 75-82 | 5 | 40 |
| 71-90 | 8-mer | SPESLLQL | 76-83 | 6 | 41 |
| 71-90 | 8-mer | PESLLQLK | 77-84 | 7 | 42 |
| 71-90 | 8-mer | ESLLQLKA | 78-85 | 8 | 43 |
| 71-90 | 8-mer | SLLQLKAL | 79-86 | 9 | 44 |
| 71-90 | 8-mer | LLQLKALK | 80-87 | 10 | 45 |
| 71-90 | 8-mer | LQLKALKP | 81-88 | 11 | 46 |
| 71-90 | 8-mer | QLKALKPG | 82-89 | 12 | 47 |
| 71-90 | 8-mer | LKALKPGV | 83-90 | 13 | 48 |
| 71-90 | 9-mer | GAADQSPES | 71-79 | 14 | 49 |
| 71-90 | 9-mer | AADQSPESL | 72-80 | 15 | 50 |
| 71-90 | 9-mer | ADQSPESLL | 73-81 | 16 | 51 |
| 71-90 | 9-mer | DQSPESLLQ | 74-82 | 17 | 52 |
| 71-90 | 9-mer | QSPESLLQL | 75-83 | 18 | 53 |
| 71-90 | 9-mer | SPESLLQLK | 76-84 | 19 | 54 |
| 71-90 | 9-mer | PESLLQLKA | 77-85 | 20 | 55 |
| 71-90 | 9-mer | ESLLQLKAL | 78-86 | 21 | 56 |
| 71-90 | 9-mer | SLLQLKALK | 79-87 | 22 | 57 |
| 71-90 | 9-mer | LLQLKALKP | 80-88 | 23 | 58 |
| 71-90 | 9-mer | LQLKALKPG | 81-89 | 24 | 59 |
| 71-90 | 9-mer | QLKALKPGV | 82-90 | 25 | 60 |
| 71-90 | 10-mer | GAADQSPESL | 71-80 | 26 | 61 |
| 71-90 | 10-mer | AADQSPESLL | 72-81 | 27 | 62 |
| 71-90 | 10-mer | ADQSPESLLQ | 73-82 | 28 | 63 |
| 71-90 | 10-mer | DQSPESLLQL | 74-83 | 29 | 64 |
| 71-90 | 10-mer | QSPESLLQLK | 75-84 | 30 | 65 |
| 71-90 | 10-mer | SPESLLQLKA | 76-85 | 31 | 66 |
| 71-90 | 10-mer | PESLLQLKAL | 77-86 | 32 | 67 |
| 71-90 | 10-mer | ESLLQLKALK | 78-87 | 33 | 68 |
| 71-90 | 10-mer | SLLQLKALKP | 79-88 | 34 | 69 |
| 71-90 | 10-mer | LLQLKALKPG | 80-89 | 35 | 70 |
| 71-90 | 10-mer | LQLKALKPGV | 81-90 | 36 | 71 |
| 101-119 | 8-mer | FLCQRPDG | 101-108 | 1 | 72 |
| 101-119 | 8-mer | LCQRPDGA | 102-109 | 2 | 73 |
| 101-119 | 8-mer | CQRPDGAL | 103-110 | 3 | 74 |
| 101-119 | 8-mer | QRPDGALY | 104-111 | 4 | 75 |
| 101-119 | 8-mer | RPDGALYG | 105-112 | 5 | 76 |
| 101-119 | 8-mer | PDGALYGS | 106-113 | 6 | 77 |
| 101-119 | 8-mer | DGALYGSL | 107-114 | 7 | 78 |
| 101-119 | 8-mer | GALYGSLH | 108-115 | 8 | 79 |
| 101-119 | 8-mer | ALYGSLHF | 109-116 | 9 | 80 |
| 101-119 | 8-mer | LYGSLHFD | 110-117 | 10 | 81 |
| 101-119 | 8-mer | YGSLHFDP | 111-118 | 11 | 82 |
| 101-119 | 8-mer | GSLHFDPE | 112-119 | 12 | 83 |
| 101-119 | 9-mer | FLCQRPDGA | 101-109 | 13 | 84 |
| 101-119 | 9-mer | LCQRPDGAL | 102-110 | 14 | 85 |
| 101-119 | 9-mer | CQRPDGALY | 103-111 | 15 | 86 |
| 101-119 | 9-mer | QRPDGALYG | 104-112 | 16 | 87 |
| 101-119 | 9-mer | RPDGALYGS | 105-113 | 17 | 88 |
| 101-119 | 9-mer | PDGALYGSL | 106-114 | 18 | 89 |
| 101-119 | 9-mer | DGALYGSLH | 107-115 | 19 | 90 |
| 101-119 | 9-mer | GALYGSLHF | 108-116 | 20 | 91 |
| 101-119 | 9-mer | ALYGSLHFD | 109-117 | 21 | 92 |
| 101-119 | 9-mer | LYGSLHFDP | 110-118 | 22 | 93 |
| 101-119 | 9-mer | YGSLHFDPE | 111-119 | 23 | 94 |
| 101-119 | 10-mer | FLCQRPDGAL | 101-110 | 24 | 95 |
| 101-119 | 10-mer | LCQRPDGALY | 102-111 | 25 | 96 |
| 101-119 | 10-mer | CQRPDGALYG | 103-112 | 26 | 97 |
| 101-119 | 10-mer | QRPDGALYGS | 104-113 | 27 | 98 |
| 101-119 | 10-mer | RPDGALYGSL | 105-114 | 28 | 99 |
| 101-119 | 10-mer | PDGALYGSLH | 106-115 | 29 | 100 |
| 101-119 | 10-mer | DGALYGSLHF | 107-116 | 30 | 101 |
| 101-119 | 10-mer | GALYGSLBFD | 108-117 | 31 | 102 |
| 101-119 | 10-mer | ALYGSLHFDP | 109-118 | 32 | 103 |
| 101-119 | 10-mer | LYGSLHFDPE | 110-119 | 33 | 104 |
| 136-170 | 8-mer | QSEAHGLP | 136-143 | 1 | 105 |
| 136-170 | 8-mer | SEAHGLPL | 137-144 | 2 | 106 |
| 136-170 | 8-mer | EAHGLPLH | 138-145 | 3 | 107 |
| 136-170 | 8-mer | AHGLPLHL | 139-146 | 4 | 108 |
| 136-170 | 8-mer | HGLPLHLP | 140-147 | 5 | 109 |
| 136-170 | 8-mer | GLPLHLPG | 141-148 | 6 | 110 |
| 136-170 | 8-mer | LPLHLPGN | 142-149 | 7 | 111 |
| 136-170 | 8-mer | PLHLPGNK | 143-150 | 8 | 112 |
| 136-170 | 8-mer | LHLPGNKS | 144-151 | 9 | 113 |
| 136-170 | 8-mer | HLPGNKSP | 145-152 | 10 | 114 |
| 136-170 | 8-mer | LPGNKSPH | 146-153 | 11 | 115 |
| 136-170 | 8-mer | PGNKSPHR | 147-154 | 12 | 116 |
| 136-170 | 8-mer | GNKSPHRD | 148-155 | 13 | 117 |
| 136-170 | 8-mer | NKSPHRDP | 149-156 | 14 | 118 |
| 136-170 | 8-mer | KSPHRDPA | 150-157 | 15 | 119 |
| 136-170 | 8-mer | SPHRDPAP | 151-158 | 16 | 120 |
| 136-170 | 8-mer | PHRDPAPR | 152-159 | 17 | 121 |
| 136-170 | 8-mer | HRDPAPRG | 153-160 | 18 | 122 |
| 136-170 | 8-mer | RDPAPRGP | 154-161 | 19 | 123 |
| 136-170 | 8-mer | DPAPRGPA | 155-162 | 20 | 124 |
| 136-170 | 8-mer | PAPRGPAR | 156-163 | 21 | 125 |
| 136-170 | 8-mer | APRGPARF | 157-164 | 22 | 126 |
| 136-170 | 8-mer | PRGPARFL | 158-165 | 23 | 127 |
| 136-170 | 8-mer | RGPARFLP | 159-166 | 24 | 128 |
| 136-170 | 8-mer | GPARFLPL | 160-167 | 25 | 129 |
| 136-170 | 8-mer | PARFLPLP | 161-168 | 26 | 130 |
| 136-170 | 8-mer | ARFLPLPG | 162-169 | 27 | 131 |
| 136-170 | 8-mer | RFLPLPGL | 163-170 | 28 | 132 |
| 136-170 | 9-mer | QSEAHGLPL | 136-144 | 29 | 133 |
| 136-170 | 9-mer | SEAHGLPLH | 137-145 | 30 | 134 |
| 136-170 | 9-mer | EAHGLPLHL | 138-146 | 31 | 135 |
| 136-170 | 9-mer | AHGLPLHLP | 139-147 | 32 | 136 |
| 136-170 | 9-mer | HGLPLHLPG | 140-148 | 33 | 137 |
| 136-170 | 9-mer | GLPLHLPGN | 141-149 | 34 | 138 |
| 136-170 | 9-mer | LPLHLPGNK | 142-150 | 35 | 139 |
| 136-170 | 9-mer | PLHLPGNKS | 143-151 | 36 | 140 |
| 136-170 | 9-mer | LHLPGNKSP | 144-152 | 37 | 141 |
| 136-170 | 9-mer | HLPGNKSPH | 145-153 | 38 | 142 |
| 136-170 | 9-mer | LPGNKSPHR | 146-154 | 39 | 143 |
| 136-170 | 9-mer | PGNKSPHRD | 147-155 | 40 | 144 |
| 136-170 | 9-mer | GNKSPHRDP | 148-156 | 41 | 145 |
| 136-170 | 9-mer | NKSPHRDPA | 149-157 | 42 | 146 |
| 136-170 | 9-mer | KSPHRDPAP | 150-158 | 43 | 147 |
| 136-170 | 9-mer | SPHRDPAPR | 151-159 | 44 | 148 |
| 136-170 | 9-mer | PHRDPAPRG | 152-160 | 45 | 149 |
| 136-170 | 9-mer | HRDPAPRGP | 153-161 | 46 | 150 |
| 136-170 | 9-mer | RDPAPRGPA | 154-162 | 47 | 151 |
| 136-170 | 9-mer | DPAPRGPAR | 155-163 | 48 | 152 |
| 136-170 | 9-mer | PAPRGPARF | 156-164 | 49 | 153 |
| 136-170 | 9-mer | APRGPARFL | 157-165 | 50 | 154 |
| 136-170 | 9-mer | PRGPARFLP | 158-166 | 51 | 155 |
| 136-170 | 9-mer | RGPARFLPL | 159-167 | 52 | 156 |
| 136-170 | 9-mer | GPARFLPLP | 160-168 | 53 | 157 |
| 136-170 | 9-mer | PARFLPLPG | 161-169 | 54 | 158 |
| 136-170 | 9-mer | ARFLPLPGL | 162-170 | 55 | 159 |
| 136-170 | 10-mer | QSEAHGLPLH | 136-145 | 56 | 160 |
| 136-170 | 10-mer | SEAHGLPLHL | 137-146 | 57 | 161 |
| 136-170 | 10-mer | EAHGLPLHLP | 138-147 | 58 | 162 |
| 136-170 | 10-mer | AHGLPLHLPG | 139-148 | 59 | 163 |
| 136-170 | 10-mer | HGLPLHLPGN | 140-149 | 60 | 164 |
| 136-170 | 10-mer | GLPLHLPGNK | 141-150 | 61 | 165 |
| 136-170 | 10-mer | LPLHLPGNKS | 142-151 | 62 | 166 |
| 136-170 | 10-mer | PLHLPGNKSP | 143-152 | 63 | 167 |
| 136-170 | 10-mer | LHLPGNKSPH | 144-153 | 64 | 168 |
| 136-170 | 10-mer | HLPGNKSPHR | 145-154 | 65 | 169 |
| 136-170 | 10-mer | LPGNKSPHRD | 146-155 | 66 | 170 |
| 136-170 | 10-mer | PGNKSPHRDP | 147-156 | 67 | 171 |
| 136-170 | 10-mer | GNKSPHRDPA | 148-157 | 68 | 172 |
| 136-170 | 10-mer | NKSPHRDPAP | 149-158 | 69 | 173 |
| 136-170 | 10-mer | KSPHRDPAPR | 150-159 | 70 | 174 |
| 136-170 | 10-mer | SPHRDPAPRG | 151-160 | 71 | 175 |
| 136-170 | 10-mer | PHRDPAPRGP | 152-161 | 72 | 176 |
| 136-170 | 10-mer | HRDPAPRGPA | 153-162 | 73 | 177 |
| 136-170 | 10-mer | RDPAPRGPAR | 154-163 | 74 | 178 |
| 136-170 | 10-mer | DPAPRGPARF | 155-164 | 75 | 179 |
| 136-170 | 10-mer | PAPRGPARFL | 156-165 | 76 | 180 |
| 136-170 | 10-mer | APRGPARFLP | 157-166 | 77 | 181 |
| 136-170 | 10-mer | PRGPARFLPL | 158-167 | 78 | 182 |
| 136-170 | 10-mer | RGPARFLPLP | 159-168 | 79 | 183 |
| 136-170 | 10-mer | GPARFLPLPG | 160-169 | 80 | 184 |
| 136-170 | 10-mer | PARFLPLPGL | 161-170 | 81 | 185 |
| 196-209 | 8-mer | MVGPSQGR | 196-203 | 1 | 186 |
| 196-209 | 8-mer | VGPSQGRS | 197-204 | 2 | 187 |
| 196-209 | 8-mer | GPSQGRSP | 198-205 | 3 | 188 |
| 196-209 | 8-mer | PSQGRSPS | 199-206 | 4 | 189 |
| 196-209 | 8-mer | SQGRSPSY | 200-207 | 5 | 190 |
| 196-209 | 8-mer | QGRSPSYA | 201-208 | 6 | 191 |
| 196-209 | 8-mer | GRSPSYAS | 202-209 | 7 | 192 |
| 196-209 | 9-mer | MVGPSQGRS | 196-204 | 8 | 193 |
| 196-209 | 9-mer | VGPSQGRSP | 197-205 | 9 | 194 |
| 196-209 | 9-mer | GPSQGRSPS | 198-206 | 10 | 195 |
| 196-209 | 9-mer | PSQGRSPSY | 199-207 | 11 | 196 |
| 196-209 | 9-mer | SQGRSPSYA | 200-208 | 12 | 197 |
| 196-209 | 9-mer | QGRSPSYAS | 201-209 | 13 | 198 |
| 196-209 | 10-mer | MVGPSQGRSP | 196-205 | 14 | 199 |
| 196-209 | 10-mer | VGPSQGRSPS | 197-206 | 15 | 200 |
| 196-209 | 10-mer | GPSQGRSPSY | 198-207 | 16 | 201 |
| 196-209 | 10-mer | PSQGRSPSYA | 199-208 | 17 | 202 |
| 196-209 | 10-mer | SQGRSPSYAS | 200-209 | 18 | 203 |

Epitopes located at amino acids 45-63 represent epitopes in region 1 of FGF21 (Pfam accession no. PF00167, start of domain sequence); epitopes located at amino acids 71-90 represent epitopes in region 3 of FGF21 (Pfam accession no. PF00167); epitopes located at amino acids 101-119 represent epitopes in region 4 of FGF21 (Pfam accession no. PF00167); epitopes located at amino acids 136-170 represent epitopes in region 2 of FGF21 (Pfam accession no. PF00167, end of domain sequence); and epitopes located at amino acids 196-209 represent epitopes in region 5 (the C-terminus) of FGF21.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the present invention.

1. A method of treating cancer or a cancer symptom in a patient in need thereof comprising administering to the patient a therapeutically effective amount of an FGF21 inhibitor.

2. A method of modulating an FGF21-related biological activity in a patient, the method comprising administering to the patient an amount of an FGF21 inhibitor effective to modulate the FGF21-related biological activity.

3. A method of modulating one or more FGF21-related activities in cancer cells that express FGF21 comprising contacting the cells with an amount of an FGF21 inhibitor effective to modulate the activities.

4. A method for inhibiting the interaction of two or more cancer cells that express FGF21 in a patient comprising administering a therapeutically effective amount of an FGF21 inhibitor to the patient.

5. The method of claim 1 wherein the FGF21 inhibitor is selected from the group consisting of:
(a) an antibody that selectively binds to an epitope in an extracellular domain (ECD) of FGF21;
(b) an isolated double-stranded RNA (dsRNA) comprising a first strand of nucleotides comprising at least 19 consecutive nucleotides of a sequence set forth in SEQ ID NOs: 1, 211 and 212, or a full complement thereof, and a second strand of nucleotides comprising a sequence substantially complementary to the first strand, wherein the dsRNA molecule is less than 627 nucleotides long;
(c) an isolated nucleic acid molecule comprising at least 10 consecutive nucleotides of a sequence at least 90% identical to a sequence selected from the group consisting of SEQ ID NOs: 1,211 and 212, or a full complement thereof;
(d) a small molecule;
(e) a mimetic;
(f) a soluble receptor; and
(g) a decoy.

6. The method of claim 1, further comprising the administration of a conventional cancer therapeutic to the patient.

7. The method of claim 1, wherein the FGF21 inhibitor reduces FGF21 expression by at least 30% as compared to a control.

8. The method of claim 1, wherein the FGF21 inhibitor causes apoptosis in at least 30% of cells in a population of cancer cells which express FGF21, as compared to a control.

9. The method of claim 3, wherein the cancer cells are endothelial cells.

10. The method of claim 3, wherein the cancer cells are colon cancer cells.

11. The method of claim 1, wherein the FGF21 inhibitor is a monoclonal antibody, a polyclonal antibody, a chimeric antibody, a human antibody, a humanized antibody, a single-chain antibody, or a Fab fragment.

12. The method of claim 11, wherein the antibody specifically binds to one or more epitopes of FGF21 selected from the group consisting of SEQ ID NOS:3-203.

13. The method of claim 11, wherein the antibody specifically binds to one or more epitopes of the FGF receptor binding domain of FGF21, said one or more epitopes selected from the group consisting of SEQ ID NOs: 3-180.

14. The method of claim 11, wherein the antibody specifically binds to one or more epitopes of the C-terminal domain of FGF21, said one or more epitopes selected from the group consisting of SEQ ID NOs:118-203.

15. The method of claim 11, wherein the antibody specifically binds to one or more epitopes in the FGF21 polypeptide encoded by exon three of an FGF21 mRNA, said one or more epitopes selected from the group consisting of SEQ ID NOs: 26-76.

16. The method of claim 11, wherein the antibody specifically binds to one or more epitopes in an immunogenic region of FGF21, said immunogenic region selected from the group consisting of immunogenic regions 1, 2, 3, 4, and 5.

17. The method of claim 1, wherein the cancer symptom is selected from the group consisting of a weight loss, anemia, abdominal pain, intestinal obstruction, blood in the stool, diarrhea, constipation, other changes in bowel habits, and colon metastases.

18. A method of identifying a cancer inhibitor, wherein the cancer is characterized by differential expression of FGF21 compared to a control, said method comprising contacting a cell expressing FGF21 with a candidate compound and determining whether an FGF21-related activity is modulated, wherein modulation of the FGF21-related activity indicates that the candidate compound is a cancer inhibitor.

19. A method of identifying a cancer inhibitor, said cancer characterized by differential expression of FGF21 compared to a control, said method comprising contacting a cell expressing FGF21 with a candidate compound and determining whether activity of a downstream marker of FGF21 is modulated, wherein modulation of the downstream marker indicates that the candidate compound is a cancer inhibitor.

20. A method of treating vascular disease or a symptom of a vascular disease in a patient in need thereof comprising administering to the patient a therapeutically effective amount of an FGF21 modulator.

21. The method of claim 20, wherein the FGF21 modulator upregulates cell proliferation in the patient by at least 30% as compared to a control.

22. The method of claim 20, wherein the FGF21 modulator upregulates one or more FGF21-related activities.

23. A method of identifying an inhibitor of a vascular disease, said method comprising contacting a cell sample expressing FGF21 with a candidate compound and determining whether an FGF21-related activity is modulated, wherein modulation of the FGF21-related activity indicates that the candidate compound is an inhibitor of vascular disease.

24. The method of claim 20, wherein the vascular disease is coronary artery disease, peripheral artery disease, atherosclerosis, abdominal aortic aneurysm, a blood clot, deep vein thrombosis, venous stasis disease, phlebitis, or varicose veins.

25. The method of claim 2 wherein the FGF21-related activity is selected from the group consisting of cell proliferation, angiogenesis, blood vessel formation, cell signaling, kinase activity, uptake of glucose into adipocytes, interactions between FGF21 and one or both of FGFR-1 or FGFR-2, phosphorylation of FGFR-1 or FGFR-2 protein, and apoptosis.

26. A composition comprising a Fibroblast Growth Factor 21 (FGF21) modulator and one or more pharmaceutically acceptable carriers, wherein the FGF21 modulator is an isolated double-stranded RNA (dsRNA); an isolated oligonucleotide comprising at least 10 consecutive nucleotides of a sequence of SEQ ID NO:1; an antibody that binds an epitope in a domain of FGF21 selected from the group consisting of the signal peptide domain and the FGF receptor binding domain; a small molecule; a mimetic; a soluble receptor; or a decoy.

27. The composition of claim 26, wherein the FGF21 modulator is an FGF21 inhibitor.

28. The composition of claim 26, wherein the FGF21 modulator is an FGF21 activator.

29. The composition of claim 26, wherein the composition modulates at least one FGF2′-related activity from the group consisting of cell proliferation, angiogenesis, blood vessel formation, cell signaling, kinase activity, glucose uptake into adipocytes, cancer cell survival, interactions between FGF21 and one or both of FGFR-1 or FGFR-2, phosphorylation of FGFR-1 or FGFR-2 protein, and apoptosis.

30. The composition of claim 26, wherein the FGF21 modulator is a monoclonal antibody, a polyclonal antibody, a chimeric antibody, a human antibody, a humanized antibody, a single-chain antibody, or a Fab fragment.

31. The composition of claim 30, wherein the antibody specifically binds to one or more epitopes of FGF21 selected from the group consisting of SEQ ID NOS:3-203.

32. The composition of claim 30, wherein the antibody specifically binds to one or more epitopes of the FGF receptor binding domain of FGF21, said one or more epitopes selected from the group consisting of SEQ ID NOs: 3-180.

33. The composition of claim 30, wherein the antibody specifically binds to one or more epitopes of the C-terminal domain of FGF21, said one or more epitopes selected from the group consisting of SEQ ID NOs:118-203.

34. The composition of claim 30, wherein the antibody specifically binds to one or more epitopes in the FGF21 polypeptide encoded by exon three of an FGF21 mRNA, said one or more epitopes selected from the group consisting of SEQ ID NOs: 26-76.

35. The composition of claim 30, wherein the antibody specifically binds to one or more epitopes in an immunogenic region of FGF21, said immunogenic region selected from the group consisting of immunogenic regions 1, 2, 3, 4, and 5.

36. The composition of claim 30, wherein the antibody is labeled.

37. The composition of claim 36, wherein the label is an enzyme, radioisotope, toxin or fluorophore.

38. The composition of claim 26, wherein the FGF21 modulator is a dsRNA molecule comprising a first strand of nucleotides comprising at least 19 consecutive nucleotides of a sequence of SEQ ID NO:1, and a second strand of nucleotides comprising a sequence substantially complementary to the first strand, wherein the dsRNA molecule is less than 627 nucleotides long.
